SEQUENCE LISTING (82 sequences)

SEQ ID NO: 1 - DNA, 833 bp, Homo sapiens
ccatggcccaggtgcagctggtggaatctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaagcagtggctggtacgatggggagttcgacccctggggccagggaaccctggtcactgtctcctcaaagctttcagggagtgcatccgccccaaaacttgaagaaggtgaattttcagaagcacgcgtatcctatgtgctgacacagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtgtggtattcggcggagggaccaagctgaccgtcctagcggccgctggatccgaacaaaagctgatctcagaagaagacctaaactcacatcaccatcaccatcactaa

SEQ ID NO: 2 - protein, 251 aa, Homo sapiens
QVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARSSGWYDGEFDPWGQGTLVTVSSKLSGSASAPKLEEGEFSEARVSYVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGVVFGGGTKLTVL

SEQ ID NO: 3 - DNA, 833 bp, Homo sapiens
ccatggcccaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactcagtgaagggccgattcaccatctccagagacaacgccaagaactcactgtatctgcaaatgaacagcctgagagccgaggacacggctgtgtattactgtgcgagaagcagtggctggtacgatggggagttcgacccctggggccagggaaccctggtcactgtctcctcaaagctttcagggagtgcatccgccccaaaacttgaagaaggtgaattttcagaagcacgcgtatcctatgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatgccgtggtattcggcggagggaccaaggtcaccgtcctagcggccgctggatccgaacaaaagctgatctcagaagaagacctaaactcacatcaccatcaccatcactaa

SEQ ID NO: 4 - protein, 251 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARSSGWYDGEFDPWGQGTLVTVSSKLSGSASAPKLEEGEFSEARVSYVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNAVVFGGGTKVTVL

SEQ ID NO: 5 - DNA, 833 bp, Homo sapiens
ccatggcccaggtgcagctggtggaatctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaagcagtggctggtacgatggggagttcgacccctggggccagggaaccctggtcactgtctcctcaaagctttcagggagtgcatccgccccaaaacttgaagaaggtgaattttcagaagcacgcgtatcctatgagctgacacagccaccctcagcgtctgggacccccgggcagagggtcaccatctcttgttctggaagcagctccaacatcggaagtaatactgtaaactggtaccagcagctcccaggagcggcccccaaactcctcatctatagtaatgatcagcggccctcaggggtccctgaccgattctctggctccaagtctggcacctcagcctccctggccatcagtgggctccagtctgaggatgaggctgattattattgtgcagcgtgggatgacagcctgaatggtgtggtattcggcggagggaccaagctgaccgtcctagcggccgctggatccgaacaaaagctgatctcagaagaagacctaaactcacatcaccatcaccatcactaa

SEQ ID NO: 6 - protein, 251 aa, Homo sapiens
QVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARSSGWYDGEFDPWGQGTLVTVSSKLSGSASAPKLEEGEFSEARVSYELTQPPSASGTPGQRVTISCSGSSSNIGSNTVNWYQQLPGAAPKLLIYSNDQRPSGVPDRFSGSKSGTSASLAISGLQSEDEADYYCAAWDDSLNGVVFGGGTKLTVL

SEQ ID NO: 7 - DNA, 833 bp, Homo sapiens
ccatggcccaggtgcagctggtgcaatctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaagcagtggctggtacgatggggagttcgacccctggggccagggaaccctggtcactgtctcctcaaagctttcagggagtgcatccgccccaaaacttgaagaaggtgaattttcagaagcacgcgtacagccagtgctgactcaacccccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtgtggtattcggcggagggaccaagctcaccgtcctagcggccgctggatccgaacaaaagctgatctcagaagaagacctaaactcacatcaccatcaccatcactaa

SEQ ID NO: 8 - protein, 251 aa, Homo sapiens
QVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARSSGWYDGEFDPWGQGTLVTVSSKLSGSASAPKLEEGEFSEARVQPVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGVVFGGGTKLTVL

SEQ ID NO: 9 - protein, 30 aa, Homo sapiens
QVQLVESGGGLVKPGGSLRLSCAASGFTFS

SEQ ID NO: 10 - protein, 120 aa, Homo sapiens
QVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARSSGWYDGEFDPWGQGTLVTVSS

SEQ ID NO: 11 - protein, 14 aa, Homo sapiens
WVRQAPGKGLEWVS

SEQ ID NO: 12 - protein, 120 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARSSGWYDGEFDPWGQGTLVTVSS

SEQ ID NO: 13 - protein, 30 aa, Homo sapiens; identical to SEQ ID NO: 9

SEQ ID NO: 14 - protein, 120 aa, Homo sapiens; identical to SEQ ID NO: 10

SEQ ID NO: 15 - protein, 14 aa, Homo sapiens
WIRQAPGKGLEWVS

SEQ ID NO: 16 - protein, 120 aa, Homo sapiens
QVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARSSGWYDGEFDPWGQGTLVTVSS

SEQ ID NO: 17 - protein, 30 aa, Homo sapiens; identical to SEQ ID NO: 9

SEQ ID NO: 18 - protein, 110 aa, Homo sapiens
SYVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGVVFGGGTKLTVL

SEQ ID NO: 19 - protein, 14 aa, Homo sapiens
WVRQAPGKGLEWVS

SEQ ID NO: 20 - protein, 110 aa, Homo sapiens
SYVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNAVVFGGGTKVTVL

SEQ ID NO: 21 - protein, 30 aa, Homo sapiens
QVQLVQSGGGLVKPGGSLRLSCAASGFTFS

SEQ ID NO: 22 - protein, 110 aa, Homo sapiens
SYELTQPPSASGTPGQRVTISCSGSSSNIGSNTVNWYQQLPGAAPKLLIYSNDQRPSGVPDRFSGSKSGTSASLAISGLQSEDEADYYCAAWDDSLNGVVFGGGTKLTVL

SEQ ID NO: 23 - protein, 14 aa, Homo sapiens
WVRQAPGKGLEWVS

SEQ ID NO: 24 - protein, 110 aa, Homo sapiens
QPVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGVVFGGGTKLTVL

SEQ ID NO: 25 - protein, 13 aa, Homo sapiens
SGSSSNIGSNTVN

SEQ ID NO: 26 - protein, 7 aa, Homo sapiens
  misc_feature (3)..(3): Xaa can be any naturally occurring amino acid
SNXQRPS

SEQ ID NO: 27 - protein, 7 aa, Homo sapiens
SNNQRPS

SEQ ID NO: 28 - protein, 7 aa, Homo sapiens
SNDQRPS

SEQ ID NO: 29 - protein, 11 aa, Homo sapiens
  misc_feature (9)..(9): Xaa can be any naturally occurring amino acid
AAWDDSLNXVV

SEQ ID NO: 30 - protein, 11 aa, Homo sapiens
AAWDDSLNAVV

SEQ ID NO: 31 - protein, 11 aa, Homo sapiens
AAWDDSLNGVV

SEQ ID NO: 32 - protein, 5 aa, Homo sapiens
  misc_feature (1)..(1): Xaa can be any naturally occurring amino acid
  misc_feature (3)..(3): Xaa can be any naturally occurring amino acid
  misc_feature (5)..(5): Xaa can be any naturally occurring amino acid
XYXMX

SEQ ID NO: 33 - protein, 5 aa, Homo sapiens
  MISC_FEATURE (1)..(1): amino acid can also be S
  MISC_FEATURE (3)..(3): amino acid can also be S
  MISC_FEATURE (5)..(5): amino acid can also be N
DYYMS

SEQ ID NO: 34 - protein, 5 aa, Homo sapiens
DYYMS

SEQ ID NO: 35 - protein, 5 aa, Homo sapiens
SYSMN

SEQ ID NO: 36 - protein, 17 aa, Homo sapiens
  misc_feature (1)..(1): Xaa can be any naturally occurring amino acid
  misc_feature (6)..(6): Xaa can be any naturally occurring amino acid
  misc_feature (8)..(8): Xaa can be any naturally occurring amino acid
XISSSXSXIYYADSVKG

SEQ ID NO: 37 - protein, 17 aa, Homo sapiens
  MISC_FEATURE (1)..(1): amino acid can also be S
  MISC_FEATURE (6)..(6): amino acid can also be G
  MISC_FEATURE (8)..(8): amino acid can also be Y
YISSSSSTIYYADSVKG

SEQ ID NO: 38 - protein, 17 aa, Homo sapiens
YISSSGSTIYYADSVKG

SEQ ID NO: 39 - protein, 17 aa, Homo sapiens
SISSSSSYIYYADSVKG

SEQ ID NO: 40 - protein, 11 aa, Homo sapiens
SSGWYDGEFDP

SEQ ID NO: 41 - protein, 32 aa, Homo sapiens
RFTISRDNAKNSLYLQMNSLRAEDTAVYYCAR

SEQ ID NO: 42 - protein, 11 aa, Homo sapiens
WGQGTLVTVSS

SEQ ID NO: 43 - protein, 32 aa, Homo sapiens; identical to SEQ ID NO: 41

SEQ ID NO: 44 - protein, 11 aa, Homo sapiens
WGQGTLVTVSS

SEQ ID NO: 45 - protein, 32 aa, Homo sapiens; identical to SEQ ID NO: 41

SEQ ID NO: 46 - protein, 11 aa, Homo sapiens
WGQGTLVTVSS

SEQ ID NO: 47 - protein, 32 aa, Homo sapiens; identical to SEQ ID NO: 41

SEQ ID NO: 48 - protein, 11 aa, Homo sapiens
WGQGTLVTVSS

SEQ ID NO: 49 - protein, 22 aa, Homo sapiens
SYVLTQPPSASGTPGQRVTISC

SEQ ID NO: 50 - protein, 15 aa, Homo sapiens
WYQQLPGTAPKLLIY

SEQ ID NO: 51 - protein, 22 aa, Homo sapiens
SYVLTQPPSASGTPGQRVTISC

SEQ ID NO: 52 - protein, 15 aa, Homo sapiens
WYQQLPGTAPKLLIY

SEQ ID NO: 53 - protein, 22 aa, Homo sapiens
SYELTQPPSASGTPGQRVTISC

SEQ ID NO: 54 - protein, 15 aa, Homo sapiens
WYQQLPGAAPKLLIY

SEQ ID NO: 55 - protein, 22 aa, Homo sapiens
QPVLTQPPSASGTPGQRVTISC

SEQ ID NO: 56 - protein, 15 aa, Homo sapiens
WYQQLPGTAPKLLIY

SEQ ID NO: 57 - protein, 32 aa, Homo sapiens
GVPDRFSGSKSGTSASLAISGLQSEDEADYYC

SEQ ID NO: 58 - protein, 10 aa, Homo sapiens
FGGGTKLTVL

SEQ ID NO: 59 - protein, 32 aa, Homo sapiens; identical to SEQ ID NO: 57

SEQ ID NO: 60 - protein, 10 aa, Homo sapiens
FGGGTKVTVL

SEQ ID NO: 61 - protein, 32 aa, Homo sapiens; identical to SEQ ID NO: 57

SEQ ID NO: 62 - protein, 10 aa, Homo sapiens
FGGGTKLTVL

SEQ ID NO: 63 - protein, 32 aa, Homo sapiens; identical to SEQ ID NO: 57

SEQ ID NO: 64 - protein, 10 aa, Homo sapiens
FGGGTKLTVL

SEQ ID NO: 65 - protein, 568 aa, Homo sapiens
MELQPPEASIAVVSIPRQLPGSHSEAGVQGLSAGDDSGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 66 - DNA, 78 bp, Homo sapiens
gcggccgctggatccgaacaaaagctgatctcagaagaagacctaaactcacatcaccatcaccatcactaatctaga

SEQ ID NO: 67 - protein, 23 aa, Homo sapiens
AAAGSEQKLISEEDLNSHHHHHH

SEQ ID NO: 68 - protein, 7 aa, Homo sapiens
KNGLVKI

SEQ ID NO: 69 - protein, 7 aa, Homo sapiens
KEELLKV

SEQ ID NO: 70 - protein, 8 aa, Homo sapiens
KFTGLSKE

SEQ ID NO: 71 - protein, 8 aa, Homo sapiens
RELPAQKW

SEQ ID NO: 72 - protein, 11 aa, Homo sapiens
KVAGSPGWVRT

SEQ ID NO: 73 - protein, 11 aa, Homo sapiens
KWWHTGALYRI

SEQ ID NO: 74 - protein, 15 aa, Homo sapiens
KVAEDEAEAAAAAKF

SEQ ID NO: 75 - protein, 13 aa, Homo sapiens
KEVELNELEPEKQ

SEQ ID NO: 76 - protein, 20 aa, Homo sapiens
KQPMNAASGAAMSLAGAEKN

SEQ ID NO: 77 - protein, 18 aa, Homo sapiens
MELQPPEASIAVVSIPRQ

SEQ ID NO: 78 - protein, 21 aa, Homo sapiens
RIGDLQAFQGHGAGNLAGLKG

SEQ ID NO: 79 - protein, 26 aa, Homo sapiens
RWALLLLFWLGWLGMLAGAVVIIVRA

SEQ ID NO: 80 - protein, 30 aa, Homo sapiens
FLVVLNFGDVGLSAGLQASDLPASASLPAK

SEQ ID NO: 81 - protein, 22 aa, Homo sapiens
LLIAGTNSSDLQQILSLLESNK

SEQ ID NO: 82 - protein, 21 aa, Homo sapiens
SLLHGDFHAFSAGPGLFSYIR

(19) United States
(12) Patent Application Publication
(10) Pub. No.: US 2008/0085241 A1
(43) Pub. Date: Apr. 10, 2008

(54) Antibody

(75) Inventors: Marike Josee Janneke Gertrude Stassar, Baerums Verk (NO); Francina Celestina Chahal, Winnipeg (CA); Jeannick Paul Cizeau, Winnipeg (CA)

Correspondence Address: ROTHWELL, FIGG, ERNST & MANBECK, P.C., 1425 K STREET, N.W., SUITE 800, WASHINGTON, DC, 20005, US

(73) Assignees: AFFITECH AS, Oslo (NO); VIVENTIA BIOTECH INC., Mississauga (CA)

(21) Appl. No.: 11/835,010

(22) Filed: Aug. 7, 2007

(30) Foreign Application Priority Data
Aug. 7, 2006 (GB) 0615662.4

(51) Int. Cl. A61K 39/395; A61K 49/00; A61P 43/00; C07K 16/18; C12N 15/00; C12N 15/11; C12N 5/06; C12P 21/04; G01N 33/574

(52) U.S. Cl. 424/9.1; 424/138.1; 424/178.1; 435/320.1; 435/325; 435/69.6; 435/7.23; 530/387.7; 530/391.3; 536/23.1

(57) ABSTRACT

The present invention provides the amino acid and nucleic acid sequences of heavy and light chain complementarity determining regions of tumor specific binding proteins. In particular, the present invention provides an antibody which comprises at least one heavy chain variable region (VH) that comprises three CDRs, wherein said heavy chain variable region comprises: (i) a heavy chain CDR1 domain that comprises the amino acid sequence SYSMN (SEQ ID NO. 35), (ii) a heavy chain CDR2 domain that comprises the amino acid sequence SISSSSSYIYYADSVKG (SEQ ID NO. 39), and (iii) a heavy chain CDR3 domain that comprises the amino acid sequence SSGWYDGEFDP (SEQ ID NO: 40), wherein said antibody is capable of specifically binding to CD98hc or fragments of CD98hc.

### Antibody

The invention relates to tumor-specific binding proteins and all uses thereof. In particular, the invention relates to antibodies or antibody fragments specific for antigens or molecules on cancer cells and to methods of use thereof.

In the year 2000, an estimated 22 million people were suffering from cancer worldwide and 6.2 millions deaths were attributed to this class of diseases. Every year, there are over 10 million new cases and this estimate is expected to grow by 50% over the next 15 years (WHO, World Cancer Report. Bernard W. Stewart and Paul Kleihues, eds. IARC Press, Lyon, 2003). Current cancer treatments are limited to invasive surgery, radiation therapy and chemotherapy, all of which cause either potentially severe side-effects, non-specific toxicity and/or traumatizing changes to ones body image and/or quality of life. Cancer can become refractory to chemotherapy reducing further treatment options and likelihood of success. The prognosis for some cancer is worse than for others and some, like lung or pancreatic cancer which are almost always fatal. In addition, some cancers with a relatively high treatment success rate, such as breast cancer, also have a very high incidence rate and, thus, remain major killers.

For instance, according to WHO, there are over 1.2 million new cases of breast cancer, worldwide, each year. Treatments consist of minimal to radical surgical removal of breast tissue and lymph nodes with radiation and chemotherapy for metastatic disease. Prognosis for localized disease is relatively good with a 5 years survival rate of around 50% but once the cancer has metastasized, it is incurable with an average survival of around 2 years. Despite improving treatment success rates, nearly 400,000 women die of breast cancer each year, the highest number of deaths to cancer in woman, ahead of deaths to lung cancer. Among the short and long term survivors, most will suffer the life-long trauma of invasive and disfiguring surgical treatment.

Another example is liver cancer, with more than half a million new cases each year and nearly the same number of deaths due to poor treatment efficacy. Hepatocellular carcinomas represent around 80% of all liver cancers and are rarely curable. Five-year survival rate is only about 10% and survival after diagnosis often less than 6 months. Although surgical resection of diseased tissue can be effective, it is not an option for the majority of cases because of the presence of cirrhosis of the liver. Hepatocellular carcinomas are largely radiation resistant and response to chemotherapy is poor.

Yet another example is that of pancreatic cancer with around 200,000 new cases per year and a very poor prognosis. In fact, the majority of patients die within a year of diagnosis and only a few percent of patients survive five years. Surgery is the only available treatment but is associated with high morbidity and complication rates because it involves not only the resection of at least part of the pancreas, but also of all of the duodenum, part of the jejunum, bile duct and gallbladder and a distal gastrectomy. In some cases, the spleen and lymph nodes are also removed.

There are many more examples of cancer where current treatments do not meet the needs of patients either due to their lack of efficacy and/or because they have high morbidity rates and severe side-effects. Those selected statistics and facts however, illustrate well the need for cancer treatments with better safety and efficacy profiles.

One of the causes for the inadequacy of current cancer treatments is their lack of selectivity for affected tissues and cells. Surgical resection always involves the removal of apparently normal tissue as a “safety margin” which can increase morbidity and risk of complications. It also always removes some of the healthy tissue that may be interspersed with tumor cells and that could potentially maintain or restore the function of the affected organ or tissue. Radiation and chemotherapy will kill or damage many normal cells due to their non-specific mode of action. This can result in serious side-effects such as severe nausea, weight loss and reduced stamina, loss of hair etc., as well as increasing the risk of developing secondary cancer later in life. Treatment with greater selectivity for cancer cells would leave normal cells unharmed thus improving outcome, side-effect profile and quality of life.

The selectivity of cancer treatment can be improved by using antibodies that are specific for molecules present only or mostly on cancer cells or which are present in higher levels on cancer cells or overexpressed in cancer cells. Such antibodies can be used to modulate the immune system and enhance the recognition and destruction of the cancer by the patient's own immune system. Most antibodies tested to date have been raised against known cancer markers in the form of mouse monoclonal antibodies, sometimes “humanized” through molecular engineering. Unfortunately, their targets can also be present in significant quantities on a subset of normal cells thus raising the risk of non-specific toxic effects. Furthermore, these antibodies are mouse proteins that are being seen by the human patient's immune system as foreign proteins. The ensuing immune reaction and antibody response can result in a loss of efficacy or in side-effects.

The inventors have used a different approach in their development of antibodies for cancer treatment. Instead of immunizing experimental animals with cancer cells or isolated cancer cell markers, they have sought out to identify only those markers that are recognized by the human immune system as sufficiently foreign to trigger the production of antibodies. This implies that the markers or antigens are usually substantially absent on normal cells and, thus, the risk of non-specific toxicity is further reduced. Thus, antibodies showing high selectivity for cancer cells/tumor cells over normal cells have been identified. Such highly selective antibodies are the subject of this patent application. In addition to being selective, preferably such antibodies are fully compatible with the patient's immune system by virtue of being fully-human proteins. The antibodies of the invention can be used for diagnostic or therapeutic uses (in particular for cancer) or as a basis for engineering other binding molecules for the target antigen. The antibodies can also be used to isolate and identify the molecule to which they bind. The role of the antigen in cancer can then be studied or the antigen can be used to develop other cancer treatments. The inventors have determined the identity of the antigen to which the antibodies of the invention bind.

The antigen is CD98hc. CD98hc is a 71 kDA type-II transmembrane protein, with the C-terminus present outside the cell. CD98hc is also known as 4F2hc, solute carrier family-3, isoform-α protein/4F2hc. It combines with different SLC-7 family proteins to form a Heteromeric Amino acid Transporter (HAT) complex that represent several of the classical mammalian amino acid transporters. HATs functionality is β-1 integrin mediated. It is broadly expressed on the basolateral membrane surface of the epithelial cells, and is known to function in cell-activation, cell-growth, cell-adhesion and when over-expressed is associated with malignant transformation. The role of CD98 in cell transformation appears to be integrin-mediated. It has been reported that the promoter region of SLC-3A2 displays a sequence homology with IL-2 and IL-2 receptor α-chain, the induction of which is important for T-cell activation. It is believed that mutations in, or defect in the regulation of CD98hc (4F2hc), encoded by SLC-3A2 would be deleterious, since CD98hc serves as a heavy subunit of six other heteromeric transporters. Thus a defect in 4F2hc could result in six defective amino acid transport activities expressed in many cell types and tissues.

CD98hc/4F2hc expression is known to be up-regulated in cancers and activated lymphocytic cells. Increased CD98hc expression has been observed in kidney, small intestine, oocytes, breast and small cell lung cancers. It was also shown that some anti-4F2hc antibodies can suppress the growth of cancer cells and 4F2hc over-expression in NIH3T3 cells resulted in their malignant transformation. It is thought that CD98hc is involved in complex cellular signaling involving multiple pathways related to cell-growth, cell adhesion and malignant transformation. To date no human antibodies to CD98hc have been identified or developed which means that human therapy is not a realistic prospect based on the work carried out to date.

The present inventors however have prepared human tumor-specific antibodies that bind to several types of tumor cells including endometrial, ovarian, prostate, pancreas, cervix, breast, lung, colon, liver and stomach. Importantly, the antibodies do not significantly bind to normal cells or tissue making them suitable candidates for diagnostics and tumor therapy.

The inventors have cloned and sequenced the antibodies and determined the sequence of the antibody light and heavy chain variable regions and complementarity determining regions 1, 2 and 3.

Accordingly, the present invention provides binding proteins, e.g. antibody molecules, which can specifically bind to CD98hc or fragments of CD98hc, or entities comprising CD98hc or fragments of CD98hc, or can inhibit or significantly reduce the function of CD98hc or prevent CD98hc interacting with its natural ligands. The present invention thus further provides binding proteins, e.g. antibody molecules, that can act as antagonists of CD98hc. Alternatively, the present invention can further provide binding proteins, e.g. antibody molecules, that can act as agonists of CD98hc. In a preferred embodiment the invention provides human binding proteins, e.g. human antibody molecules, with these properties.

In addition, the binding proteins of the invention are tumor specific in that the binding proteins bind to one or more types of tumor cell, but the binding to normal tissue is insignificant or not prohibitive for diagnostic or therapeutic applications, e.g. the binding protein binds to normal tissue which will never come into contact with the binding proteins of the invention, e.g. normal tissue in the brain, which the binding proteins will not reach because they do not cross the blood brain barrier. Preferably, the binding proteins bind to one or more types of tumor cell in a way that or at a level that is effective for diagnostic or therapeutic purposes (e.g. show significant and measurable binding to tumor cells).

Amino acid and/or DNA sequences of antibody molecules which can specifically bind to CD98hc, their V_{H} and V_{L} domains and CDRs are set forth in the various SEQ ID Nos. listed herein.

In one embodiment the present invention provides a binding protein comprising a heavy chain CDR3 domain comprising the amino acid sequence of SEQ ID No.40 or a sequence substantially homologous thereto, and/or comprising a light chain CDR3 domain comprising the amino acid sequence of SEQ ID NO: 29 or a sequence substantially homologous thereto.

In a preferred embodiment, said binding protein further comprises a heavy chain CDR2 domain comprising the amino acid sequence of SEQ ID No.36 or a sequence substantially homologous thereto, and/or further comprises a light chain CDR2 domain comprising the amino acid sequence of SEQ ID NO: 26 or a sequence substantially homologous thereto.

Alternatively, said binding proteins comprising the above defined CDR3 domains further comprise a heavy chain CDR1 domain comprising the amino acid sequence of SEQ ID No.32 or a sequence substantially homologous thereto, and/or further comprise a light chain CDR1 domain comprising the amino acid sequence of SEQ ID NO: 25 or a sequence substantially homologous thereto.

In more preferred embodiments the above described CDR1 and CDR 2 domains are present.

Viewed alternatively, the present invention provides a binding protein comprising a heavy chain CDR2 domain and/or a light chain CDR2 domain as defined above. Said binding protein optionally further comprises a heavy chain CDR3 domain and/or a light chain CDR3 domain as defined above and/or further comprises a heavy chain CDR1 domain and/or a light chain CDR1 domain as defined above.

Viewed yet alternatively, the present invention provides a binding protein comprising a heavy chain CDR1 domain and/or a light chain CDR1 domain as defined above. Said binding protein optionally further comprises a heavy chain CDR3 domain and/or a light chain CDR3 domain as defined above and/or further comprises a heavy chain CDR2 domain and/or a light chain CDR2 domain as defined above.

In all the embodiments of the present invention as described herein, X in an amino acid sequence represents a variable amino acid. In addition, preferred embodiments of SEQ ID NO: 26 are SEQ ID NO: 27 or 28 (more preferably SEQ ID NO: 27); preferred embodiments of SEQ ID NO: 29 are SEQ ID No: 30 or 31 (more preferably SEQ ID NO: 31), preferred embodiments of SEQ ID NO: 32 are SEQ ID NOs: 33, 34 or 35 (more preferably SEQ ID NO: 35) and preferred embodiments of SEQ ID NO: 36 are SEQ ID NOs: 37, 38 or 39 (more preferably SEQ ID NO: 39).

A yet further embodiment of the invention provides a binding protein comprising one or more of the CDRs of the invention or sequences substantially homologous thereto as set out herein. Preferred binding proteins comprise one or more of the CDRs selected from the group consisting of SEQ ID NOS. 25, 26, 29, 32, 33, 36, 37 and 40 or a sequence substantially homologous thereto. Most preferred binding proteins comprise one or more of the CDRs selected from the group consisting of SEQ ID NOS. 25, 27, 28, 30, 31, 34, 35, 38, 39 and 40, or a sequence substantially homologous thereto.

Thus, in preferred embodiments the binding protein comprises a light chain CDR 1 comprising the amino acid sequence SGSSSNIGSNTVN (SEQ ID NO. 25), or a sequence substantially homologous thereto; or comprises a light chain CDR 2 comprising the amino acid sequence SNXQRPS (SEQ ID NO:26) (where X is a variable amino acid, preferably N or D, i.e. preferably SNNQRPS (SEQ ID NO:27) or SNDQRPS (SEQ ID NO:28), more preferably N, i.e. SNNQRPS), or sequences substantially homologous thereto; or comprises a light chain CDR3 comprising the amino acid sequence AAWDDSLNXVV (SEQ ID NO: 29) (where X is a variable amino acid, preferably A or G, i.e. preferably AAWDDSLNAVV (SEQ ID NO: 30) or AAWDDSLNGVV (SEQ ID NO:31), more preferably G, i.e. AAWDDSLNGW), or sequences substantially homologous thereto. In especially preferred embodiments the binding protein comprises a light chain CDR 1 comprising the amino acid sequence SGSSSNIGSNTVN (SEQ ID NO. 25), or a sequence substantially homologous thereto.

In other preferred embodiments the binding protein comprises a heavy chain CDR1 comprising the amino acid sequence XYXMX (SEQ ID NO. 32) (where X is a variable amino acid), preferably D/S Y Y/S M S/N (SEQ ID NO. 33), more preferably DYYMS (SEQ ID NO:34) or SYSMN (SEQ ID NO:35), most preferably SYSMN, or sequences substantially homologous thereto; or comprises a heavy chain CDR2 comprising the amino acid sequence XISSSXSXIYYADSVKG (SEQ ID NO. 36) (where X is a variable amino acid), preferably Y/S ISSS S/G S T/Y IYYADSVKG (SEQ ID NO. 37), more preferably YISSSGSTIYYADSVKG (SEQ ID NO:38) or SISSSSSYIYYADSVKG (SEQ ID NO:39), most preferably SISSSSSYIYYADSVKG, or sequences substantially homologous thereto; or comprises a heavy chain CDR3 comprising the amino acid sequence SSGWYDGEFDP (SEQ ID NO: 40), or a sequence substantially homologous thereto. As outlined above, in especially preferred embodiments the binding protein comprises a heavy chain CDR3 comprising the amino acid sequence SSGWYDGEFDP (SEQ ID NO: 40), or a sequence substantially homologous thereto.

Preferred binding proteins comprise two or more of the light chain CDRs of the invention or sequences substantially homologous thereto as described above. Especially preferred binding molecules comprise 3 of the light chain CDRs of the invention or sequences substantially homologous thereto as described above (i.e. one of each of the light chain CDR1 and CDR2 and CDR3).

Other preferred binding proteins comprise two or more of the heavy chain CDRs of the invention or sequences substantially homologous thereto as described above. Especially preferred binding molecules comprise 3 of the heavy chain CDRs of the invention or sequences substantially homologous thereto as described above (i.e. one of each of the heavy chain CDR1 and CDR2 and CDR3). Most preferred binding proteins comprise 3 of the light chain CDRs of the invention or sequences substantially homologous thereto as described above and 3 of the heavy chain CDRs of the invention or sequences substantially homologous thereto as described above.

Especially preferred binding molecules comprise a heavy chain CDR1 domain of SEQ ID NO: 32, a CDR2 domain of SEQ ID NO: 36, and a CDR3 domain of SEQ ID NO: 40, or sequences substantially homologous thereto; and/or comprise a light chain CDR1 domain of SEQ ID NO: 25, a CDR2 domain of SEQ ID NO: 26, and a CDR 3 domain of SEQ ID NO: 29, or sequences substantially homologous thereto.

Further preferred embodiments provide binding proteins comprising a V_{H} domain which comprises one or more of the heavy chain CDRs of the invention or sequences substantially homologous thereto, as described above, and/or a V_{L} domain which comprises one or more of the light chain CDRs of the invention or sequences substantially homologous thereto, as described above.

Preferred light chain variable regions (V_{L} domains) comprise 2 or more of the light chain CDRs of the invention or sequences substantially homologous thereto, as described above. Especially preferred V_{L} domains comprise 3 of the light chain CDRs of the invention or sequences substantially homologous thereto as described above (i.e. one of each of CDR1, CDR2 and CDR3). Preferred heavy chain variable regions (V_{H} domains) comprise 2 or more of the heavy chain CDRs of the invention or sequences substantially homologous thereto, as described above. Especially preferred V_{H} domains comprise 3 of the heavy chain CDRs of the invention or sequences substantially homologous thereto as described above (i.e. one of each of CDR1, CDR2 and CDR3). Most preferred binding proteins comprise 3 of the light chain CDRs of the invention or sequences substantially homologous thereto as described above and 3 of the heavy chain CDRs of the invention or sequences substantially homologous thereto as described above.

Preferred CDR domains and combinations thereof comprising the V_{H} or V_{L} domains are described elsewhere herein. However, an especially preferred V_{L} domain (or binding protein) comprises the CDR1 of SGSSSNIGSNTVN (SEQ ID NO. 25), or sequences substantially homologous thereto.

In a further embodiment, the V_{L} domain (or binding protein) comprises the CDR regions of SGSSSNIGSNTVN (CDR 1) (SEQ ID NO: 25) and/or SNXQRPS (CDR 2) (SEQ ID NO: 26) and/or AAWDDSLNXW (CDR 3) (SEQ ID NO: 29), or sequences substantially homologous thereto. (In such embodiments preferred X residues are as defined above).

In a further embodiment, the V_{L} domain (or binding protein) comprises the complementarity determining regions of SEQ ID NOS: 25, 27 and/or 30, or sequences substantially homologous thereto. In another embodiment, the V_{L} domain (or binding protein) comprises the complementarity determining regions of SEQ ID NOS: 25, 28 and/or 31, or sequences substantially homologous thereto. In a further embodiment, the V_{L} domain (or binding protein) comprises the complementarity determining regions of SEQ ID NOS: 25, 27 and/or 31, or sequences substantially homologous thereto.

An especially preferred V_{H} domain (or binding protein) comprises the CDR3 of SSGWYDGEFDP (SEQ ID NO. 40), or sequences substantially homologous thereto.

In a further embodiment, the V_{H} domain (or binding protein) comprises the CDR regions of D/S Y Y/S M S/N (CDR 1) (SEQ ID NO: 33) and/or XISSSXSXIYYADSVKG (CDR 2) (SEQ ID NO: 36) and/or SSGWYDGEFDP (CDR 3) (SEQ ID NO: 40), or sequences substantially homologous thereto. (In such embodiments preferred X residues are as defined above).

In a further embodiment, the V_{H} domain (or binding protein) comprises the complementarity determining regions of SEQ ID NOS: 34, 38 and/or 40, or sequences substantially homologous thereto. In another embodiment, the V_{H} domain (or binding protein) comprises the complementarity determining regions of SEQ ID NOS: 35, 39 and/or 40, or sequences substantially homologous thereto.

Any combination of the above discussed V_{L} and V_{H} domains can be present in the binding proteins of the invention. Thus, a preferred binding protein of the invention comprises a V_{L} domain which comprises the CDR regions of SGSSSNIGSNTVN (CDR 1) (SEQ ID NO: 25) and/or SNXQRPS (CDR 2) (SEQ ID NO: 26) and/or AAWDDSLNXW (CDR 3) (SEQ ID NO: 29), or sequences substantially homologous thereto, and a V_{H} domain which comprises the CDR regions of D/S Y Y/S M S/N (CDR 1) (SEQ ID NO: 33) and/or XISSSXSXIYYADSVKG (CDR 2) (SEQ ID NO: 36) and/or SSGWYDGEFDP (CDR 3) (SEQ ID NO: 40), or sequences substantially homologous thereto. (In such embodiments preferred X residues are as defined above).

Other preferred binding proteins comprise (i) the complementarity determining regions of SEQ ID NOS: 25, 27 and 30 (more preferably as part of a V_{L} domain), or sequences substantially homologous thereto, and/or the complementarity determining regions of SEQ ID NOS: 34, 38 and 40 (more preferably as part of a V_{H} domain), or sequences substantially homologous thereto; or (ii) the complementarity determining regions of SEQ ID NOS: 25, 28 and 31 (more preferably as part of a V_{L} domain), or sequences substantially homologous thereto, and/or the complementarity determining regions of SEQ ID NOS: 35, 39 and 40 (more preferably as part of a V_{H} domain), or sequences substantially homologous thereto; or (iii) the complementarity determining regions of SEQ ID NOS: 25, 27 and 31 (more preferably as part of a V_{L} domain), or sequences substantially homologous thereto, and/or the complementarity determining regions of SEQ ID NOS: 35, 39 and 40 (more preferably as part of a V_{H} domain), or sequences substantially homologous thereto.

A yet further embodiment of the invention provides a binding protein comprising a V_{H} domain which has the amino acid sequence of SEQ ID NO. 10, 12, 14 or 16, or a sequence substantially homologous thereto, and/or a V_{L} domain which has the amino acid sequence of SEQ ID NO. 18, 20, 22 or 24, or a sequence substantially homologous thereto.

Preferred embodiments of the invention provide a binding protein comprising a V_{H} domain which has the amino acid sequence of SEQ ID NO. 10 and a V_{L} domain which has the amino acid sequence of SEQ ID NO. 18, or sequences substantially homologous thereto, or a binding protein comprising a V_{H} domain which has the amino acid sequence of SEQ ID NO. 12 and a V_{L} domain which has the amino acid sequence of SEQ ID NO. 20, or sequences substantially homologous thereto, or a binding protein comprising a V_{H} domain which has the amino acid sequence of SEQ ID NO. 14 and a V_{L} domain which has the amino acid sequence of SEQ ID NO. 22, or sequences substantially homologous thereto, or a binding protein comprising a V_{H} domain which has the amino acid sequence of SEQ ID NO. 16 and a V_{L} domain which has the amino acid sequence of SEQ ID NO. 24, or sequences substantially homologous thereto.

In a yet further embodiment the present invention provides a binding protein comprising the amino acid sequence of SEQ ID No. 2 (also referred to herein as clone EJ212/076-Cl10), 4 (also referred to herein as clone VB2-169), 6 (also referred to herein as clone VB2-170) or 8 (also referred to herein as clone VB2-187), or comprising a fragment thereof, or a sequence substantially homologous thereto.

The term “binding protein” as used herein refers to proteins that specifically bind to another substance. In particular, binding proteins of the invention specifically bind to CD98hc or fragments of CD98hc, or to entities comprising CD98hc or fragments of CD98hc, or can inhibit or significantly reduce the function of CD98hc or can prevent CD98hc interacting with its natural ligands. In a preferred embodiment binding proteins are human proteins. In a further preferred embodiment, binding proteins are antibodies or antibody fragments or comprise antibodies or antibody fragments. The binding proteins of the invention can thus be made up of a single polypeptide chain or multiple polypeptide chains which assemble or associate to form the binding protein.

When it is desired to use the binding proteins of the invention for tumor therapy or diagnosis, then the binding proteins are also preferably tumor specific in that the binding proteins bind to one or more types of tumor cell or sample, but the binding to normal cells or tissue is insignificant or undetectable or not prohibitive for diagnostic or therapeutic applications, e.g. the binding protein binds to normal tissue which will never come into contact with the binding proteins of the invention, e.g. normal tissue in the brain, which the binding proteins will not reach because they do not cross the blood brain barrier. Preferably, the binding proteins bind to one or more types of tumor cell or sample in a way that or at a level that is effective for diagnostic or therapeutic purposes (e.g. show significant and measurable binding to one or more types of tumor cells or samples, but show weaker binding, preferably no significant binding, to one or more types of normal cells or samples). Appropriate ways of assessing such tumor specificity are well known and described in the art, for example by FACS or immunohistochemical profiling, in which generally the binding of a binding protein to several tumor cell lines or samples is compared to the binding of the protein to normal cell lines or samples and the finding of a measurable or significant difference (increase) in binding to tumor versus normal cells or samples indicates tumor specificity. Exemplary normal and tumor cell lines or samples which can be used are described in the Examples. Preferably the significant difference in binding is statistically significant, preferably with a probability value of <0.05. Appropriate methods of determining statistical significance are well known and documented in the art and any of these may be used.

Thus, the term “normal cells” is used herein to refer to non-cancerous cells. This term encompasses healthy cells which occur naturally within the human body, in particular peripheral red blood cells or granulocytes. The term “do not significantly bind to normal cells” should be understood such that any binding of the binding protein to normal cells does not prohibit the use of said binding protein for therapeutic or diagnostic purposes. Thus, by “insignificant” binding to normal cells is meant that the binding of the binding protein to normal cells is weaker than its binding to one or more tumor cells. Some cross-reaction with normal cells may thus occur, but this level of binding can be considered to be “background” binding. For therapeutic or diagnostic purposes the main consideration is that the binding protein must bind more strongly to one or more types of tumor cells than to any healthy cells with which the binding protein may come into contact during the therapeutic or diagnostic application.

The term “tumor specific” should be interpreted such that the binding of the binding protein to the tumor cells is specific enough to allow the use of said binding protein for therapeutic or diagnostic purposes. The skilled person can easily determine if any given binding protein is tumour specific by comparing the binding strength to the target tumor cell with the binding strength to one or more types of normal cells, e.g. peripheral red blood cells or granulocytes.

Preferably, the binding proteins have a binding affinity for one or more types of cancer cells which corresponds to a Km of less than 1 μM, more preferably of less than 500, 400 or 300 nM, even more preferably of less than 200, 190, 180, 170, 160, 150, 140, 130, 120, 1 10, or 100 nM, most preferably of less than 90, 80, 70, 60, 50, 40, 30, 20, 10, 5 or 1 nM. For example, the binding affinity may be 5.6×10⁻⁸ M or less, or 9.0×10⁻⁹ M or less. Any appropriate method of determining Km may be used. However, preferably the Km is determined by testing various concentrations of the binding protein against a fixed number of target cells in vitro to establish a saturation curve, for example using the Lineweaver-Burk method. A suitable assay is described in Example 3 for illustrative purposes.

The binding proteins preferably have a Km for one or more types of tumor cells which is at least 50% less, more preferably at least 1, 2, 3, 4 or 5 orders of magnitude lower than the Km for one or more types of non-cancerous or normal cells, e.g. PBL cells or granulocytes, when binding affinity is assayed under comparable conditions, in particular using the same dosage of binding protein and cells in each assay.

The term “human” as used herein in connection with antibody molecules and binding proteins refers to binding proteins having variable (e.g. V_{H}, V_{L}, CDR or FR regions) and/or constant antibody regions derived from or corresponding to sequences found in humans, e.g. in the human germline or somatic cells. The “human” binding proteins of the invention further include amino acid residues not encoded by human sequences, e.g. mutations introduced by random or site directed mutations in vitro (in particular mutations which involve conservative substitutions or mutations in a small number of residues of the binding protein, e.g. in 1, 2, 3, 4 or 5 of the residues making up one or more of the CDRs of the binding protein). In addition, the human binding proteins of the present invention include proteins comprising human consensus sequences identified from human sequences.

In addition, the human binding proteins of the present invention are not limited to combinations of V_{H}, V_{L}, CDR or FR regions which are themselves found in combination in human antibody molecules. Thus, the human binding proteins of the invention can include or correspond to combinations of such regions which do not necessarily exist naturally in humans.

The term “antibody” or “antibody molecule” as used herein refers to immunoglobulin molecules or other molecules which comprise an antigen binding domain.

The term “antibody” or “antibody molecule” as used herein is thus intended to include whole antibodies (e.g. IgG, IgA, IgE, IgM, or IgD), monoclonal antibodies, polyclonal antibodies, and chimeric antibodies. Antibody fragments which comprise an antigen binding domain are also included. The term “antibody fragment” as used herein is intended to include any appropriate antibody fragment that displays antigen binding function, for example Fab, Fab′, F(ab′)₂, scFv, Fv, dsFv, ds-scFv, Fd, dAbs, TandAbs dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art.

The antibodies or antibody fragments can be produced naturally or can be wholly or partially synthetically produced. Thus the antibody may be from any appropriate source, for example recombinant sources and/or produced in transgenic animals or transgenic plants. Thus, the antibody molecules can be produced in vitro or in vivo.

Preferably the antibody or antibody fragment comprises an antibody light chain variable region (V_{L}) and an antibody heavy chain variable region (V_{H}) which generally comprise the antigen binding site. In certain embodiments, the antibody or antibody fragment comprises all or a portion of a heavy chain constant region, such as an IgG1, IgG2, IgG3, IgG4, IgA1, IgA2, IgE, IgM or IgD constant region. Preferably, the heavy chain constant region is an IgG1 heavy chain constant region. Furthermore, the antibody or antibody fragment can comprise all or a portion of a kappa light chain constant region or a lambda light chain constant region. Preferably, the light chain constant region is a lambda light chain constant region. All or part of such constant regions may be produced naturally or may be wholly or partially synthetic. Appropriate sequences for such constant regions are well known and documented in the art.

The term “fragment” as used herein refers to fragments of biological relevance, e.g. fragments which can contribute to or enable antigen binding, e.g. form part or all of the antigen binding site, or can contribute to the inhibition or reduction in function of the antigen or can contribute to the prevention of the antigen interacting with its natural ligands. Preferred fragments thus comprise a heavy chain variable region (V_{H} domain) and/or a light chain variable region (V_{L} domain) of the antibodies of the invention. Other preferred fragments comprise one or more of the heavy chain complementarity determining regions (CDRs) of the antibodies of the invention (or of the V_{H} domains of the invention), or one or more of the light chain complementarity determining regions (CDRs) of the antibodies of the invention (or of the V_{L} domains of the invention). When used in the context of a nucleic acid molecule, the term “fragment” includes a nucleic acid molecule encoding a fragment as described herein.

In embodiments where the binding proteins of the invention comprise a fragment of any of the defined sequences (for example comprise a fragment of SEQ ID Nos 2, 4, 6 or 8), e.g. are binding proteins comprising V_{H} and/or V_{L} domains of the invention, or are binding proteins comprising one or more CDRs of the invention, then these regions/domains are generally separated within the binding protein so that each region/domain can perform its biological function and so that the contribution to antigen binding is retained. Thus, the V_{H} and V_{L} domains may be separated by appropriate scaffold sequences/linker sequences and the CDRs may be separated by appropriate framework regions such as those found in naturally occurring antibodies. Thus, the V_{H}, V_{L} and individual CDR sequences of the invention can be provided within or incorporated into an appropriate framework or scaffold to enable antigen binding. Such framework sequences or regions can correspond to naturally occurring framework regions, FR1, FR2, FR3 and/or FR4, as appropriate to form an appropriate scaffold, or can correspond to consensus framework regions, for example identified by comparing various naturally occurring framework regions. Alternatively, non-antibody scaffolds or frameworks, e.g. T cell receptor frameworks can be used.

Appropriate sequences which can be used for framework regions are well known and documented in the art and any of these may be used. Preferred sequences for framework regions are one or more of the framework regions making up the V_{H} and/or V_{L} domains of the invention, i.e. one or more of the framework regions disclosed in SEQ ID Nos 2, 4, 6 or 8 (or in Table 6), or framework regions substantially homologous thereto, and in particular framework regions which allow the maintenance of antigen specificity, for example framework regions which result in substantially the same or the same 3D structure of the binding protein. In preferred embodiments, all four FR regions of SEQ ID NOS: 2, 4, 6 or 8 (also shown in Table 6) (or FR regions substantially homologous thereto) are found in the binding proteins of the invention.

In addition, although preferred binding proteins of the invention are made up of V_{H}, V_{L} or CDRs of the invention, it should be noted that the binding proteins of the invention also encompass one or more V_{H}, V_{L} or CDRs of the invention in combination with other V_{H}, V_{L} or CDRs not of the invention provided that the binding specificity for antigen (CD98hc), or the ability to inhibit or significantly reduce the function of CD98hc or prevent CD98hc interacting with its natural ligands, or the tumor specific properties of the binding proteins of the invention as outlined above are still present.

The term “heavy chain complementarity determining region” as used herein refers to regions of hypervariability within the heavy chain variable region (V_{H} domain) of an antibody molecule. The heavy chain variable region has three complementarity determining regions termed heavy chain complementarity determining region 1, heavy chain complementarity determining region 2 and heavy chain complementarity determining region 3 from the amino terminus to carboxy terminus. The heavy chain variable region also has four framework regions (FR1, FR2, FR3 and FR4 from the amino terminus to carboxy terminus). These regions separate the CDRs.

The term “heavy chain variable region” (V_{H} domain) as used herein refers to the variable region of a heavy chain of an antibody molecule.

The term “light chain complementarity determining region” as used herein refers to regions of hypervariability within the light chain variable region (V_{L} domain) of an antibody molecule. Light chain variable regions have three complementarity determining regions termed light chain complementarity determining region 1, light chain complementarity determining region 2 and light chain complementarity determining region 3 from the amino terminus to the carboxy terminus. The light chain variable region also has four framework regions (FR1, FR2, FR3 and FR4 from the amino terminus to carboxy terminus). These regions separate the CDRs.

The term “light chain variable region” (V_{L} domain) as used herein refers to the variable region of a light chain of an antibody molecule.

It should be noted that the Kabat nomenclature is followed herein, where necessary, in order to define the positioning of the CDRs (Kabat, E. A., Wu, T. T., Perry, H. M., Gottesman, K. S., and Foeller, C. (1991) Sequences of Proteins of Immunological Interest, 5th Ed, Public Health Service, National Institutes of Health, Bethesda, Md.).

The term “light chain CDR” and “heavy chain CDR” is used herein for the purpose of nomenclature and does not necessitate that the binding protein has a definable light and/or heavy chain, or even that the particular CDR is found on a heavy or light chain if such identifiable parts are present.

Nucleic acid molecules comprising sequences encoding the binding proteins of the invention as defined above, or nucleic acid molecules substantially homologous thereto, form a yet further aspect of the invention. Preferred nucleic acid molecules are as defined in SEQ ID NOS: 1, 3, 5 or 7, or nucleic acid molecules substantially homologous thereto.

Fragments of the binding proteins of the invention as defined above, or sequences substantially homologous thereto, form a yet further aspect of the invention.

Accordingly, the invention provides a polypeptide comprising or consisting of a V_{L} domain of the invention as defined above, or a sequence substantially homologous thereto, or a polypeptide comprising or consisting of a V_{H} domain of the invention as defined above, or a sequence substantially homologous thereto.

Accordingly, the invention further provides a polypeptide comprising or consisting of one or more of the CDR regions of the invention as defined above, or sequences substantially homologous thereto.

When more than one CDR region is present, preferred combinations are also as described above.

Nucleic acid molecules comprising sequences encoding such fragments of the binding proteins of the invention, or nucleic acid molecules substantially homologous thereto, form a yet further aspect of the invention. Preferred nucleic acid sequences encoding such fragments (e.g. V_{H} domains, V_{L} domains, and individual CDRs) can be found in SEQ ID NOS: 1, 3, 5 or 7.

The term “substantially homologous” as used herein in connection with an amino acid or nucleic acid sequence includes sequences having at least 50%, preferably at least 60%, more preferably at least 70%, most preferably at least 80%, and even more preferably at least 85%, 90%, 92%, 95%, 96%, 97%, 98% or 99%, sequence identity to the amino acid or nucleic acid sequence disclosed. Substantially homologous sequences of the invention thus include single or multiple base or amino acid alterations (additions, substitutions, insertions or deletions) to the sequences of the invention. At the amino acid level preferred substantially homologous sequences contain only 1, 2, 3, 4 or 5, preferably 1, 2 or 3, more preferably 1 or 2, altered amino acids, in one or more of the framework regions and/or one or more of the CDRs making up the sequences of the invention. Preferably said alterations are conservative amino acid substitutions. In sequences where variable amino acids are represented by “X”, then preferably said alterations are found at residues which are not “X” residues.

The substantially homologous nucleic acid sequences also include nucleotide sequences that hybridize to the nucleic acid sequences disclosed (or their complementary sequences), e.g. hybridize to nucleotide sequences encoding one or more of the light chain or heavy chain CDRs of the invention, the light or heavy chain variable regions of the invention, or the binding proteins of the invention (or hybridize to their complementary sequences), under at least moderately stringent hybridization conditions.

The term “substantially homologous” also includes modifications or chemical equivalents of the amino acid and nucleotide sequences of the present invention that perform substantially the same function as the proteins or nucleic acid molecules of the invention in substantially the same way. For example any substantially homologous binding protein should retain the ability to specifically bind to the CD98hc antigen and preferably to the same epitope thereof as recognized by the binding protein in question, for example, the same epitope or antigen recognised by the CDR domains of the invention or the V_{H} and V_{L} domains of the invention as described herein. Binding to the same epitope/antigen can be readily tested by methods well known and described in the art, e.g. using binding assays, e.g. a competition assay such as that described below and in Example 6.

Thus, a person skilled in the art will appreciate that binding assays can be used to find other antibodies and antibody fragments with the same binding specificities as the antibodies and antibody fragments of the invention. As exemplified, below, a competition binding assay can be used to find such other antibodies.

Before a competition assay is performed using flow cytometry, the minimal concentration of antibody of the invention (Ab1) that gives maximal binding against a fixed number of tumor cells is determined. A total of 10⁶ cells are harvested from exponentially growing cultures and incubated with various antibody concentrations for 1 hr at 4° C. The cells are washed and incubated with a suitable detection antibody for an additional hour at 4° C. After washing, the cells are analyzed by flow cytometry. For each test antibody, a saturation curve is generated from the data by plotting median fluorescence against the antibody concentration.

For the competition assay, tumor cells are prepared as above and treated in duplicate with a fixed concentration of antibody (Ab1). The fixed concentration is the minimal concentration of antibody that generates maximal binding against a fixed number of tumor cells as determined above. Immediately following the addition of the Ab1, varying concentrations of the potential inhibitory antibody (Ab2) is added to each tube and the mixture incubated for 1 hr at 4° C. Both the percent inhibition and change over maximum median fluorescence are calculated by subtracting the background fluorescence (PBS-5% FCS) from the median fluorescence reading for each test sample (Ab1+Ab2). The result is then divided by the median fluorescence of Ab1 alone (maximal binding) minus the background (see below). The percent of inhibition result is obtained by multiplying by 100. The mean of the replicates along with their respective standard error is plotted against antibody concentration. The following formula is used to calculate the percent inhibition:
PI=[(MF(_{Ab1+Ab2)-}MF_{Bgd})/(MF_{Ab1}−MF_{Bgd})]×100
where PI=percent inhibition; MF(_{Ab1+Ab2})=median fluorescence measured for Ab1+Ab2 mixture; and MF_{Bgd}=background median fluorescence with PBS-5% FCS.

Accordingly, the invention provides a binding protein capable of binding an antigen on a tumor cell wherein the binding protein can be identified by a method comprising:
(1) incubating a fixed number of tumor cells with a minimal concentration of a binding protein of the invention, preferably an antibody or antibody fragment (Ab1) that generates maximal binding against the fixed number of tumor cells and measuring median fluorescence of Ab1 (MF_{Ab1});
(2) testing two or more concentrations of a test binding protein (Ab2) by adding Ab2 to the Ab1 and tumor cells, and measuring median fluorescence (MF_{(Ab1+Ab2)});
(3) measuring background median fluorescence (MF_{bgd});
(4) calculating PI, wherein
   PI=[(MF(_{Ab1+Ab2})−MF_{Bgd})/(MF_{Ab1}−MF_{Bgd})]×100; and
(5) comparing the PI to a control PI value;
wherein, a PI that has a statistically significant difference from the control PI indicates that the test binding protein is capable of binding the antigen on the tumor cell. An appropriate control PI would be obtained by performing the competition experiment with an irrelevant antibody, i.e. one that does not bind to the tumor cells. Appropriate irrelevant antibodies to use in this regard would be readily determined by a person skilled in the art depending on the tumor cell in question. Preferably the statistically significant difference has a probability value of <0.05. Appropriate methods of determining statistical significance are well known and documented in the art and any of these may be used. The invention thus further provides a method of identifying a binding protein capable of binding an antigen on a tumour cell, said method comprising the method steps as outlined above.

Any substantially homologous binding protein should also preferably retain the tumor specificity as described elsewhere herein, e.g. retain the ability to bind to tumor tissue without significantly binding to normal tissue.

Substantially homologous sequences of proteins of the invention include, without limitation, conservative amino acid substitutions, or for example alterations which do not effect the V_{H}, V_{L} or CDR domains of the binding proteins, e.g. include scFv antibodies where a different linker sequence is used or binding proteins where tag sequences or other 30 components are added which do not contribute to the binding of antigen, or alterations to convert one type or format of antibody molecule or fragment to another type or format of antibody molecule or fragment (e.g. conversion from Fab to scFv or vice versa), or the conversion of an antibody molecule to a particular class or subclass of antibody molecule (e.g. the conversion of an antibody molecule to IgG or a subclass thereof, e.g. IgG1 or IgG3).

A “conservative amino acid substitution”, as used herein, is one in which the amino acid residue is replaced with another amino acid residue having a similar side chain. Families of amino acid residues having similar side chains have been defined in the art, including basic side chains (e.g., lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Homology may be assessed by any convenient method. However, for determining the degree of homology between sequences, computer programs that make multiple alignments of sequences are useful, for instance Clustal W (Thompson, J. D., D. G. Higgins, et al. (1994). “CLUSTAL W: Improving the sensitivity of progressive multiple sequence alignment through sequence weighting, position-specific gap penalties and weight matrix choice”. Nucleic Acids Res 22: 4673-4680). If desired, the Clustal W algorithm can be used together with BLOSUM 62 scoring matrix (Henikoff S. and Henikoff J. G., 1992, Proc. Natl. Acad. Sci. USA 89: 10915-10919) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Other methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Programs that compare and align pairs of sequences, like ALIGN (E. Myers and W. Miller, “Optical Alignments in Linear Space”, CABIOS (1988) 4: 11-17), FASTA (W. R. Pearson and D. J. Lipman (1988), “Improved tools for biological sequence analysis”, PNAS 85:2444-2448, and W. R. Pearson (1990) “Rapid and sensitive sequence comparison with FASTP and FASTA” Methods in Enzymology 183:63-98) and gapped BLAST (Altschul, S. F., T. L. Madden, et al. (1997). “Gapped BLAST and PSI-BLAST: a new generation of protein database search programs”. Nucleic Acids Res. 25: 3389-3402), BLASTP, BLASTN, or GCG (Devereux et al., Nucleic Acids Res., 1984, 12: 387) are also useful for this purpose. Furthermore, the Dali server at the European Bioinformatics institute offers structure-based alignments of protein sequences (Holm, J. of Mol. Biology, 1993, Vol. 233: 123-38; Holm, Trends in Biochemical Sciences, 1995, Vol 20: 478-480; Holm, Nucleic Acid Research, 1998, Vol. 26: 316-9).

By way of providing a reference point, sequences according to the present invention having 50%, 60%, 70%, 80%, 90%, 95% homology etc. may be determined using the ALIGN program with default parameters (for instance available on Internet at the GENESTREAM network server, IGH, Montpellier, France).

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log10 [Na+])+0.41 (%(G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5×sodium chloride/sodium citrate (SSC)/5×Denhardt's solution/1.0% SDS at Tm−5° C. based on the above equation, followed by a wash of 0.2×SSC/0.1% SDS at 60° C. Moderately stringent hybridization conditions include a washing step in 3×SSC at 42° C.

By way of further example, sequences which “hybridize” are those sequences binding (hybridising) under non-stringent conditions (e.g. 6×SSC, 50% formamide at room temperature) and washed under conditions of low stringency (e.g. 2×SSC, room temperature, more preferably 2×SSC, 42° C.) or conditions of higher stringency (e.g. 2×SSC, 65° C.) (where SSC=0.15M NaCl, 0.015M sodium citrate, pH 7.2).

It is understood, however, that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 1989, Vol.3.

Generally speaking, sequences which hybridise under conditions of high stringency are preferred, as are sequences which, but for the degeneracy of the code, would hybridise under high stringency conditions.

The polypeptide, binding protein and nucleic acid molecules of the invention are generally isolated molecules insofar as they are not present in situ within a human or animal body or a tissue sample derived from a human or animal body. The sequences may however correspond to or be substantially homologous to sequences as found in a human or animal body. Thus, the term “isolated” as used herein in reference to nucleic acid molecules or sequences and proteins or polypeptides, refers to such molecules when isolated from or substantially free of their natural environment, e.g. isolated from the human or animal body (if indeed they occur naturally), or refers to such molecules when produced by a technical process, i.e. includes recombinant and synthetically produced molecules.

Thus, when used in connection with a nucleic acid molecule, such a term may refer to a nucleic acid substantially free of material with which it is naturally associated such as other nucleic acids/genes or polypeptides. This term may also refer to a nucleic acid substantially free of cellular material or culture medium when produced by recombinant DNA techniques, or substantially free of chemical precursors, or other chemicals when chemically synthesized. An isolated nucleic acid may also be substantially free of sequences which naturally flank the nucleic acid (i.e. sequences located at the 5′ and 3′ ends of the nucleic acid) from which the nucleic acid is derived or sequences which have been made to flank the nucleic acid (e.g. tag sequences or other sequence which have no therapeutic value) by for example genetic engineering.

Thus, when used in connection with a protein or polypeptide molecule such as light chain complementarity regions 1, 2 and 3, heavy chain complementarity regions 1, 2 and 3, light chain variable regions, heavy chain variable regions, and binding proteins of the invention, the term “isolated” may refer to a protein substantially free of cellular material or other proteins from the source from which it is derived. In some embodiments, particularly where the protein is to be administered to humans or animals, such isolated proteins are substantially free of culture medium when produced by recombinant techniques, or chemical precursors or other chemicals when chemically synthesized. Such isolated proteins may also be free of flanking sequences such as those described above for the isolated nucleic acid molecules.

The term “nucleic acid sequence” or “nucleic acid molecule” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid molecules may be double stranded or single stranded. The nucleic acid molecules may be wholly or partially synthetic or recombinant.

A person skilled in the art will appreciate that the proteins and polypeptides of the invention, such as the light and heavy complementarity determining regions, the light and heavy chain variable regions, binding proteins, antibodies and antibody fragments, and immunoconjugates, may be prepared in any of several ways well known and described in the art, but are most preferably prepared using recombinant methods.

Accordingly, the nucleic acid molecules of the present invention may be cloned or synthesised by any appropriate method and may be incorporated in a known manner into an appropriate expression vector which ensures good expression of the proteins of the invention. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the invention and regulatory sequences selected on the basis of the host cells to be used for expression, which are operatively linked to the nucleic acid molecule. Operatively linked is intended to mean that the nucleic acid is linked to regulatory sequences in a manner which allows expression of the nucleic acid.

The invention therefore contemplates a recombinant expression vector containing a nucleic acid molecule of the invention, or a fragment thereof, and the necessary regulatory sequences for the transcription and translation of the protein sequence encoded by the nucleic acid molecule of the invention.

Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors of the invention may also contain a selectable marker gene which facilitates the selection of host cells transformed or transfected with a recombinant molecule of the invention. Examples of selectable marker genes are genes encoding a protein such as neomycin and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the invention and in particular to determine the effect of a mutation on expression and phenotype. It will be appreciated that selectable markers can be introduced on a separate vector from the nucleic acid of interest.

The recombinant expression vectors may also contain genes which encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMaI (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. The term “transformed host cell” as used herein is intended to also include cells capable of glycosylation that have been transformed with a recombinant expression vector of the invention. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of eukaryotic host cells and prokaryotic cells. For example, the proteins of the invention may be expressed in yeast cells or mammalian cells. Other suitable host cells can be found in Goeddel, Gene Expression Technology: Methods in Enzymology 185, Academic Press, San Diego, Calif. (1991). In addition, the proteins of the invention may be expressed in prokaryotic cells, such as Escherichia coli (Zhang et al., Science 303(5656): 371-3 (2004)).

Yeast and fungi host cells suitable for carrying out the present invention include, but are not limited to Saccharomyces cerevisiae, the genera Pichia or Kluyveromyces and various species of the genus Aspergillus. Examples of vectors for expression in yeast S. cerevisiae include pYepSec1 (Baldari. et al., Embo J. 6:229-234 (1987)), pMFa (Kurjan and Herskowitz, Cell 30:933-943 (1982)), pJRY88 (Schultz et al., Gene 54:113-123 (1987)), and pYES2 (Invitrogen Corporation, San Diego, Calif.). Protocols for the transformation of yeast and fungi are well known to those of ordinary skill in the art (see Hinnen et al., Proc. Natl. Acad. Sci. USA 75:1929 (1978); Itoh et al., J. Bacteriology 153:163 (1983), and Cullen et al. (Biol Technology 5:369 (1987)).

Mammalian cells suitable for carrying out the present invention include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (Seed, B., Nature 329:840 (1987)) and pMT2PC (Kaufman et al., EMBO J. 6:187-195 (1987)).

Given the teachings provided herein, promoters, terminators, and methods for introducing expression vectors of an appropriate type into plant, avian, and insect cells may also be readily accomplished. For example, within one embodiment, the proteins of the invention may be expressed from plant cells (see Sinkar et al., J. Biosci (Bangalore) 11:47-58 (1987), which reviews the use of Agrobacterium rhizogenes vectors; see also Zambryski et al., Genetic Engineering, Principles and Methods, Hollaender and Setlow (eds.), Vol. VI, pp. 253-278, Plenum Press, New York (1984), which describes the use of expression vectors for plant cells, including, among others, PAPS2022, PAPS2023, and PAPS2034)

Insect cells suitable for carrying out the present invention include cells and cell lines from Bombyx, Trichoplusia or Spodotera species. Baculovirus vectors available for expression of proteins in cultured insect cells (SF 9 cells) include the pAc series (Smith et al., Mol. Cell Biol. 3:2156-2165 (1983)) and the pVL series (Lucklow, V. A., and Summers, M. D., Virology 170:31-39 (1989)). Some baculovirus-insect cell expression systems suitable for expression of the recombinant proteins of the invention are described in PCT/US/02442.

Alternatively, the proteins of the invention may also be expressed in non-human transgenic animals such as, rats, rabbits, sheep and pigs (Hammer et al. Nature 315:680-683 (1985); Palmiter et al. Science 222:809-814 (1983); Brinster et al. Proc. Natl. Acad. Sci. USA 82:4438-4442 (1985); Palmiter and Brinster Cell 41:343-345 (1985) and U.S. Pat. No. 4,736,866). Thus, the present invention also provides a transgenic non-human animal comprising a nucleic acid molecule or vector of the invention or expressing a binding protein of the invention.

The proteins of the invention may also be prepared by chemical synthesis using techniques well known in the chemistry of proteins such as solid phase synthesis (Merrifield, J. Am. Chem. Assoc. 85:2149-2154 (1964); Frische et al., J. Pept. Sci. 2(4): 212-22 (1996)) or synthesis in homogenous solution (Houbenweyl, Methods of Organic Chemistry, ed. E. Wansch, Vol. 15 I and II, Thieme, Stuttgart (1987)).

N-terminal or C-terminal fusion proteins comprising the proteins of the invention conjugated with other molecules, such as proteins may be prepared by fusing, through recombinant techniques. The resultant fusion proteins contain a protein of the invention fused to the selected protein or marker protein as described herein. The proteins of the invention may also be conjugated to other proteins by known techniques. For example, the proteins may be coupled using heterobifunctional thiol-containing linkers as described in WO 90/10457, N-succinimidyl-3-(2-pyridyldithio-proprionate) or N-succinimidyl-5 thioacetate. Examples of proteins which may be used to prepare fusion proteins or conjugates include cell binding proteins such as immunoglobulins, hormones, growth factors, lectins, insulin, low density lipoprotein, glucagon, endorphins, transferrin, bombesin, asialoglycoprotein glutathione-S-transferase (GST), hemagglutinin (HA), and truncated myc.

Accordingly, the invention provides a recombinant expression vector comprising one or more of the nucleic acid sequences of the invention or one or more of the nucleic acid sequences that encode the proteins of the invention (such as the light and heavy chain complementarity determining regions, the light and heavy chain variable regions, or the binding proteins, such as antibodies and antibody fragments).

Further, the invention provides a host cell comprising one or more of the recombinant expression vectors or one or more of the nucleic acid sequences of the invention, or a host cell expressing one or more of the proteins of the invention (such as the light and heavy chain complementarity determining regions, the light and heavy chain variable regions, or the binding proteins, such as antibodies and antibody fragments).

A yet further aspect of the invention provides a method of producing a protein of the present invention comprising a step of culturing the host cells of the invention. Preferred methods comprise the steps of (i) culturing a host cell comprising one or more of the recombinant expression vectors or one or more of the nucleic acid sequences of the invention under conditions suitable for the expression of the protein; and optionally (ii) isolating the protein from the host cell or from the growth medium/supernatant. Such methods of production may also comprise a step of purification of the protein product and/or formulating the product into a composition including at least one additional component, such as a pharmaceutically acceptable carrier or excipient.

In embodiments when the protein of the invention is made up of more than one polypeptide chain (e.g. certain fragments such as Fab fragments), then all the polypeptides are preferably expressed in the host cell, either from the same or a different expression vector, so that the complete proteins, e.g. binding proteins of the invention, can assemble in the host cell and be isolated therefrom.

The binding proteins of the invention have specificity for the CD98hc antigen. Thus, the binding proteins of the invention can be used to detect CD98hc in vivo or in vitro. Thus, the binding proteins of the invention can target the body sites which express the CD98hc antigen, whereupon the binding protein can act at the target site (e.g. target tissue, organ or cells). Furthermore, the binding proteins of the invention can be conjugated to other entities and used to target these other entities to body sites which express the CD98hc antigen. (Where the binding protein is an antibody molecule then such conjugates are also referred to as immunoconjugates). Such other entities could be labels or other detectable moieties, in which case these conjugate molecules would be useful for in vivo or in vitro diagnosis or imaging of body sites, in particular body sites afflicted with cancer. Appropriate labels and detectable moieties are discussed elsewhere herein. Alternatively the binding proteins of the invention could be conjugated to biologically active molecules or medically relevant agents such as toxins, enzymes, drugs, pre drugs, pro drugs or other small molecule compounds, or nucleic acid molecules (e.g. antisense molecules), in which case these molecules would be useful for targeted therapy, for example by targeting the drug, toxin or enzyme, etc., to cells or body sites where the CD98hc antigen is expressed. Such biologically active molecules or medically relevant agents may be in an active form or in a form which is to be activated, for example in the body. In particular, such molecules could be used for targeting cancer cells.

Binding protein conjugates are thus preferred binding proteins of the invention. Preferred binding proteins to be used in the conjugates are full length (whole) antibodies, F(ab′)₂, Fab or scFv.

Methods for conjugating such other entities to the binding proteins of the invention are well known and described in the art and an appropriate method can readily be selected depending on the nature of the binding protein and the other entity to be conjugated. Thus, the other entities can be conjugated to the binding proteins of the invention either directly or via an intermediate, e.g. an appropriate linker. The conjugation might for example be covalent or non-covalent (e.g. the other entities can be conjugated to the binding protein via the formation of a complex with the binding protein or more conveniently with an intermediate linking entity such as a chemical group or a peptide tag). Such binding as a complex is for example appropriate for many radioisotopes.

In such embodiments, the binding proteins (e.g. the antibody or antibody fragment), together with the conjugated entity, could be included or incorporated in an artificial membrane, forming e.g. an artificial particle such as a micelle, liposome or nanoparticle. These particles would be guided to a target body site by virtue of the binding protein and could then fuse with the cells at the target site (or be internalized—see below), thereby releasing the conjugated entity, e.g. the biologically active molecules or medically relevant agents, from the inside of the artificial particle into the target cell, e.g. a tumor cell. Again, methods of incorporating molecules into such artificial membranes are well known and described in the art.

An interesting property of some of the binding proteins (e.g. antibodies or antibody fragments) of the invention is their ability to be internalized into the cells to which they become bound. Thus, in a preferred embodiment, the binding proteins of the invention are capable of being internalized. This property is particularly advantageous for use in such conjugates as the biologically active molecule or medically relevant agent should be internalized with the antibody molecules. Preferred binding proteins for use in this regard are binding proteins of the invention as defined elsewhere herein, in particular binding proteins comprising SEQ ID Nos: 2, 4, 6, 8,10, 12,14,16,18, 20, 22 or 24 (or sequences substantially homologous thereto) and especially preferably SEQ ID NOs: 4, 6,12, 14, 20 or 22 (or sequences substantially homologous thereto). In general, the internalization of a binding protein is dependent on the antigen it binds to. Thus, providing the binding protein interacts with CD98hc with a sufficient affinity such that the binding protein does not dissociate from the CD98hc before the CD98hc is internalized, then the binding protein will also be internalized. This is clearly advantageous for certain embodiments.

Thus, it can be seen that a yet further aspect of the invention provides the binding proteins (e.g. binding protein conjugates) or other proteins of the invention as defined herein for use in therapy, diagnosis or imaging.

In addition, the invention provides compositions comprising the binding proteins of the invention, such as antibodies and antibody fragments, optionally together with one or more pharmaceutically acceptable excipient, carrier, diluent, buffer or stabilizer.

Such compositions can be used in any of aspects of the invention described herein where a binding protein is used, e.g. can be used in any of the methods, uses or kits as described herein.

A yet further aspect of the invention provides the use of the binding proteins (e.g. binding protein conjugates) or other proteins of the invention as defined herein in the manufacture of a composition or medicament for use in therapy, imaging or diagnosis.

Methods of treatment of a subject comprising the administration of an effective amount of a binding protein (e.g. binding protein conjugate) or other protein of the invention as defined herein to a subject, or to a sample (e.g. a blood sample) removed from a subject and which is subsequently returned to the subject, provide yet further aspects of the invention.

The in vivo methods as described herein are generally carried out in a mammal. Any mammal may be treated, for example humans and any livestock, domestic or laboratory animal. Specific examples include mice, rats, pigs, cats, dogs, sheep, rabbits, cows and monkeys. Preferably however the mammal is a human.

The terms “therapy” or “treatment” as used herein include prophylactic therapy, which may result in the prevention of disease. The terms “therapy” and “treatment” include combatting or cure of disease but also include the controlling, reduction or alleviation of disease or one or more of the symptoms associated therewith.

An “effective amount” as used herein can refer to a therapeutically effective amount or a prophylactically effective amount depending on the nature of the treatment. A therapeutically effective amount can be considered to be an amount necessary (at appropriate dosages and administration regimes) to achieve the desired therapeutic result. A prophylactically effective amount can be considered to be an amount necessary (at appropriate dosages and administration regimes) to achieve the desired prophylactic result. As indicated below, the amounts are likely to vary depending on the weight, age and sex of the patient, the severity of the disease and the ability of the binding protein to elicit a desired response in the individual.

The compositions of the present invention can be formulated according to any of the conventional methods known in the art and widely described in the literature. Thus, the active ingredient (i.e. the binding protein) may be incorporated, optionally together with other active substances (examples of which are as described below), with one or more conventional pharmaceutically acceptable carriers, diluents and/or excipients, etc., appropriate for the particular use for a composition, to produce conventional preparations which are suitable or can be made suitable for administration. They may be formulated as liquids, as semi-solids or as solids, e.g. liquid solutions, dispersions, suspensions, tablets, pills, powders, sachets, cachets, elixirs, emulsions, syrups, ointments, liposomes, suppositories, and the like. The preferred form depends on the intended mode of administration and therapeutic application. Preferably the composition comprising the binding protein of the invention is prepared in a form of an injectable or infusible solution.

The preferred mode of administration is parenteral, e.g. intraperitoneal, intravenous, subcutaneous, intramuscular, intracavity or transdermal, although any other appropriate mode may be used, for example oral administration. Intravenous injection or infusion is especially preferred. Any appropriate site of administration may be used. For example they may be administered locally and directly at the site where action is required or may be attached or otherwise associated, e.g. conjugated, with entities which will facilitate the targeting to an appropriate location in the body. Any physiologically compatible carrier, excipient, diluent, buffer or stabilizer can be used in the compositions of the invention. Examples of suitable carriers, excipients, diluents, buffers and stabilizers include one or more of water, saline, phosphate buffered saline, dextrose, glycerol, ethanol and the like, as well as combinations thereof. In some cases isotonic agents, e.g. sugars, polyalcohols (e.g. mannitol, sorbitol), or sodium chloride may be included. The compositions may additionally include lubricating agents, wetting agents, emulsifying agents, suspending agents, preserving agents, sweetening agents, flavouring agents, and the like. The compositions of the invention may be formulated so as to provide quick, sustained or delayed release of the active ingredient after administration to the subject by employing procedures well known in the art. As described above, preferably the composition is in a form suitable for injection and suitable carriers may be present at any appropriate concentration, but exemplary concentrations are from 1% to 20% and preferably from 5% to 10%.

Therapeutic compositions typically must be sterile and stable under conditions of manufacture and storage. Appropriate ways of achieving such sterility and stability are well known and described in the art.

In addition to a binding protein of the invention, the composition may further comprise one or more other active ingredients such as other agents which are useful for treating diseases with which CD98hc is associated or in which CD98hc activity is detrimental, e.g. cancers. Suitable additional active agents for inclusion in a composition that is to be used in the treatment of mammals will be known to a person skilled in the art and can be selected depending on the nature of the disease which is to be treated by the composition. Suitable additional agents include antibodies which bind to other targets, cytokines, and chemical agents, e.g. standard chemotherapeutics (small molecule drugs) or drugs controlling side effects.

Suitable doses of the binding protein of the invention and the other active ingredients (if included) will vary from patient to patient and will also depend on the nature of the particular disease. Preferably, said dosages constitute a therapeutically effective amount or a prophylactically effective amount, depending on the nature of the treatment involved. Suitable doses can be determined by the person skilled in the art or the physician in accordance with the weight, age and sex of the patient and the severity of the disease. The ability of the binding protein to elicit a desired response in the individual will also be a factor. Exemplary daily doses are: 0.1 to 250 mg/kg, preferably 0.1 to 200 or 100 mg/kg, more preferably 1 to 50 or 1 to 10 mg/kg, of the active ingredient. This can be administered as a single unit dose or as multiple unit doses administered more than once a day. It is to be noted however that appropriate dosages may vary depending on the patient and that for any particular subject, specific dosage regimes should be adjusted over time according to the individual needs of the patient. Thus, the dosage ranges set forth herein are to be regarded as exemplary and are not intended to limit the scope or practice of the claimed composition.

Yet further aspects are methods of diagnosis or imaging of a subject comprising the administration of an appropriate amount of a binding protein (e.g. binding protein conjugate) or other protein of the invention as defined herein to the subject and detecting the presence and/or amount and/or the location of the binding protein or other protein of the invention in the subject.

Appropriate diseases to be treated, imaged or diagnosed in accordance with the above described uses and methods include any disease associated with molecules recognised by the proteins of the invention, in particular diseases in which CD98hc is associated or plays a role, e.g. diseases associated with the presence or overexpression of CD98hc or where inhibition of CD98hc activity might be advantageous.

The binding proteins of the invention bind selectively to cancer cells or molecules internalized by cancer cells, and not significantly to normal cells. Therefore the binding proteins can be used in the diagnosis, imaging or therapy of cancer. As stated above, the inventors have shown that the binding proteins of the invention bind to CD98hc. Thus, the specificity of the binding proteins for tumor antigens makes it useful in the diagnosis, imaging or therapy of cancer.

In one embodiment of the invention, cancer includes, without limitation, cervical cancer, uterine cancer, ovarian cancer, pancreatic cancer, kidney cancer, gallbladder cancer, liver cancer, head and neck cancer, squamous cell carcinoma, gastrointestinal cancer, breast cancer (such as carcinoma, ductal, lobular, and nipple), prostate cancer, testicular cancer, lung cancer, non-small cell lung cancer, non-Hodgkin's lymphoma, multiple myeloma, leukemia (such as acute lymphocytic leukemia, chronic lymphocytic leukemia, acute myelogenous leukemia, and chronic myelogenous leukemia), brain cancer, neuroblastoma, sarcomas, colon cancer, rectum cancer, stomach cancer, bladder cancer, pancreatic cancer, endometrial cancer, plasmacytoma, lymphoma, and melanoma. In a preferred embodiment, the cancer includes, without limitation, endometrial cancer, ovarian cancer, prostate cancer, pancreatic cancer, cervical cancer, breast cancer, lung cancer, colon cancer, liver cancer, stomach cancer, head and neck cancer and skin cancer. Especially preferred are head and neck cancer or skin cancer.

In a preferred embodiment, the binding proteins are antibodies or antibody fragments of the invention.

In addition, cancer cells may be evaluated to determine their susceptibility to the treatment methods of the invention by, for example, obtaining a sample of the cancer cells from a subject and determining the ability of the cancer cells in the sample to bind to the binding proteins of the invention, preferably antibodies or antibody fragments.

Accordingly, the present invention includes diagnostic methods, agents, and kits that can be used by themselves, or prior to, during or subsequent to the therapeutic method of the invention in order to determine whether or not cancer cells are present that express the antigen and can bind to the binding proteins of the invention, preferably antibodies and antibody fragments.

In one embodiment, the invention provides a method of diagnosing disease, preferably cancer, in a mammal comprising the step of:
(1) contacting a test sample taken from said mammal with any one or more of the binding proteins of the invention.

In a further embodiment, the invention provides a method of diagnosing disease, preferably cancer, in a mammal comprising the steps of:
(1) contacting a test sample taken from said mammal with one or more of the binding proteins of the invention;
(2) measuring the presence and/or amount and/or location of binding protein-antigen complex in the test sample; and, optionally
(3) comparing the presence and/or amount of binding protein-antigen complex in the test sample to a control.

In one embodiment, the antigen is CD98hc.

In the above methods, said contacting step is carried out under conditions that permit the formation of a binding protein-antigen complex. Appropriate conditions can readily be determined by a person skilled in the art.

In the above methods any appropriate test sample may be used, for example biopsy cells, tissues or organs suspected of being affected by cancer, histological sections or blood.

In the above methods the presence of an amount of binding protein-antigen complex in the test sample would be indicative of the presence of cancer cells. For a positive diagnosis to be made, generally the amount of binding protein-antigen complex in the test sample is greater than, preferably significantly greater than, the amount found in an appropriate control sample. More preferably, the significantly greater levels are statistically significant, preferably with a probability value of <0.05. Appropriate methods of determining statistical significance are well known and documented in the art and any of these may be used.

Appropriate control samples could be readily chosen by a person skilled in the art, for example, in the case of diagnosis of a particular disease, an appropriate control would be a sample from a subject that did not have that disease.

For use in the diagnostic or imaging applications, the binding proteins of the invention, preferably antibodies or antibody fragments, may be labeled with a detectable marker such as a radio-opaque or radioisotope, such as ³H, ¹⁴C, ³²P, ³⁵S, ¹²³I, ¹²⁵I, ¹³¹I; a radioactive emitter (e.g. α, β or γ emitters); a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion; or a chemical moiety such as biotin which may be detected by binding to a specific cognate detectable moiety, e.g. labelled avidin/streptavidin. As described above, methods of attaching a label to a binding protein, such as an antibody or antibody fragment, are known in the art. Such detectable markers allow the presence, amount or location of binding protein-antigen complexes in the test sample to be examined.

Another aspect of the invention is a method of diagnosing disease, preferably cancer, in a mammal comprising the steps of:
(1) measuring the presence and/or amount of antibodies of the invention in a test sample taken from said mammal; and optionally
(2) comparing the presence and/or amount of antibodies of the invention in the test sample to a control.

In one embodiment, the amount of antibodies of the invention is measured by measuring the amount of antibodies of the invention in the test sample, for example by ELISA using CD98hc as antigen. In another embodiment, the amount of antibodies of the invention is measured by measuring the expression levels of nucleic acids encoding the antibodies of the invention in the test sample, for example by RT-PCR.

The invention also includes diagnostic or imaging agents comprising the binding proteins of the invention (e.g. antibodies or antibody fragments) attached to a label that produces a detectable signal, directly or indirectly. Appropriate labels are described elsewhere herein.

The invention further includes kits comprising one or more of the binding proteins or compositions of the invention or one or more of the nucleic acid molecules encoding the binding proteins of the invention, or one or more recombinant expression vectors comprising the nucleic acid sequences of the invention, or one or more host cells comprising the recombinant expression vectors or nucleic acid sequences of the invention. Preferably said kits are for use in the methods and uses as described herein, e.g. the therapeutic, diagnostic or imaging methods as described herein, or are for use in the in vitro assays or methods as described herein. The binding protein in such kits may preferably be a binding protein conjugate as described elsewhere herein, e.g. may be conjugated to a detectable moiety. Preferably said kits comprise instructions for use of the kit components, for example in diagnosis. Preferably said kits are for diagnosing cancer and optionally comprise instructions for use of the kit components to diagnose cancer.

The invention further includes a kit for diagnosing cancer comprising one or more of the binding proteins of the invention and optionally instructions for the use thereof to diagnose the cancer. The invention also includes a kit for diagnosing cancer comprising a binding protein, preferably an antibody or antibody fragment that binds to CD98hc, and optionally instructions for the use thereof to diagnose cancer.

The binding proteins as defined herein may also be used as molecular tools for in vitro or in vivo applications and assays. As the binding proteins have an antigen binding site, these can function as members of specific binding pairs and these molecules can be used in any assay where the particular binding pair member is required. For example, in the embodiments when the binding proteins are antibodies or antibody fragments which can bind particular antigens such as CD98hc these molecules can be used in any assay requiring an antibody with a specificity for that particular antigen, for example can be used in any assay where detection of CD98hc is required or desired.

Thus, yet further aspects of the invention provide a reagent which comprises a binding protein as defined herein and the use of such binding proteins as molecular tools, for example in in vitro or in vivo assays, e.g. in in vitro or in vivo assays to detect CD98 hc.

CD98hc is a 71 kDA type-Il transmembrane protein, with the C-terminus present outside the cell. CD98hc is also known as solute carrier family-3, isoform-α protein/4F2hc. It combines with different SLC-7 family proteins to form a Heteromeric Amino acid Transporter (HAT) complex that represent several of the classical mammalian amino acid transporters. HATs functionality is β-1 integrin mediated. It is broadly expressed on the basolateral membrane surface of the epithelial cells, and is known to function in cell-activation, cell-growth, cell-adhesion and when over-expressed is associated with malignant transformation. It has been reported that the promoter region of SLC-3A2 displays a sequence homology with IL-2 and IL-2 receptor α-chain, the induction of which is important for T-cell activation. It is believed that mutations in, or defect in the regulation of CD98hc (4F2hc), encoded by SLC-3A2 would be deleterious, since CD98hc serves as a heavy subunit of six other heteromeric transporters. Thus a defect in 4F2hc could result in six defective amino acid transport activities expressed in many cell types and tissues.

CD98hc/4F2hc expression is known to be up-regulated in cancers and activated lymphocytic cells. Increased CD98hc expression has been observed in kidney, small intestine, oocytes, breast and small cell lung cancers. The role of CD98 in cell transformation appears to be integrin-mediated. The dynamic regulation of integrin affinity for ligands in response to cellular signals is central to integrin function. It is thought that CD98hc is involved in complex cellular signaling involving multiple pathways related to cell-growth, cell adhesion and malignant transformation.

Regardless of the mechanism, the binding proteins of the invention could be used to modulate the signaling of CD98hc involving cell-growth, cell adhesion and malignant transformation.

Accordingly, the invention includes the use of the binding proteins of the invention to modulate the activity of CD98hc. For example, the binding proteins of the invention can be used to interfere with or inhibit CD98hc activity. The binding proteins of the invention may also be used to enhance CD98hc activity. As an example, the binding proteins may be used to induce apoptosis of cells.

The binding proteins of the invention may also be used to produce further binding proteins which are specific for CD98hc. Such uses involve for example the modification or mutation of, for example the addition, deletion, substitution or insertion of, one or more amino acids in the amino acid sequence of a parent binding protein to form a new binding protein, wherein said parent binding protein is one of the binding proteins of the invention as defined elsewhere herein, and testing the resulting new binding protein to identify binding proteins specific for CD98hc. Such methods can be used to form multiple new binding proteins which can all be tested for their ability to bind CD98hc. Preferably said addition, deletion, substitution or insertion of one or more amino acids takes place in one or more of the CDR domains.

Such modification or mutation to a parent binding protein can be carried out in any appropriate manner using techniques well known and documented in the art, for example by carrying out methods of random or directed mutagenesis. If directed mutagenesis is to be used then one strategy to identify appropriate residues for mutagenesis utilizes the resolution of the crystal structure of the binding protein-antigen complex, e.g. the Ab-Ag complex, to identify the key residues involved in the antigen binding (Davies D. R., Cohen G. H. 1996. Interactions of protein antigens with antibodies. Proc Natl. Acad. Sci. U.SA. 93, 7-12). Subsequently, those residues can be mutated to enhance the interaction. Alternatively, one or more amino acid residues can simply be targeted for directed mutagenesis and the effect on binding to CD98hc assessed.

Random mutagenesis can be carried out in any appropriate way, e.g. by error-prone PCR, chain shuffling or mutator E. coli strains.

Thus, one or more of the V_{H} domains of the invention can be combined with a single V_{L} domain or a repertoire of V_{L} domains from any appropriate source and the resulting new binding proteins tested to identify binding proteins specific for CD98hc. Conversely, one or more of the V_{L} domains of the invention can be combined with a a single V_{H} domain or repertoire of V_{H} domains from any appropriate source and the resulting new binding proteins tested to identify binding proteins specific for CD98hc.

Similarly, one or more, or preferably all three CDRs of the V_{H} and/or V_{L} domains of the invention can be grafted into a single V_{H} and/or V_{L} domain or a repertoire of V_{H} and/or V_{L} domains, as appropriate, and the resulting new binding proteins tested to identify binding proteins specific for CD98hc.

The targeted mutations of the CDRs, especially CDR3 of the light and/or heavy chains, have been shown to be an effective technique for increasing antibody affinity and are preferred. Preferably, blocks of 3 to 4 amino acids of the CDR3 or specific regions called “hot-spots” are targeted for mutagenesis.

“Hot spots” are the sequences where somatic hypermutation takes place in vivo (Neuberger M. S and Milstein C. 1995. Somatic hypermutation. Curr. Opin. Immunol. 7, 248-254). The hotspot sequences can be defined as consensus nucleotide sequences in certain codons. The consensus sequence is the tetranucleotide, RGYW, in which R can be either A or G, Y can be C or T and W can be either A or T (Neuberger M. S and Milstein C. 1995. Somatic hypermutation. Curr. Opin. Immunol. 7, 248-254). In addition, the serine residues encoded by the nucleotides AGY are predominantly present in the CDRs regions of the variable domain over those encoded by TCN corresponding to a potential hot-spot sequences (Wagner S. D., Milstein C. and Neuberger M. S. 1995. Codon bias targets mutation. Nature, 376, 732).

Thus, the nucleotide sequence of the CDRs of the heavy and light chains of each antibody of the invention can be scanned for the presence of the hot-spot sequences and AGY codons. The identified hot-spots of the CDR regions of the light and heavy chain can then optionally be compared to the germinal sequences of the heavy and light chains using the International ImMunoGen Tics database (IMGT, http://imgt.cines.fr/textes/vquest/) (Davies D. R., Padlan E. A. and Sheriff S. 1990. Antibody-antigen complexes. Annu. Rev. Biochem. 59, 439-473). A sequence, identical to the germ line, suggest that somatic mutation has not occurred; therefore random mutations can be introduced mimicking the somatic events occurring in vivo or alternatively, site directed mutagenesis can be carried out, e.g. at the hot spots and/or AGY codons. In contrast, a different sequence shows that some somatic mutations have already occurred. It will remain to be determined if the in vivo somatic mutation was optimal.

Preferred hot-spots for mutation are those that code for exposed amino acids and preferably those that encode amino acids which form part of the antigen binding sites. Other preferred hot-spots for mutation are those that code for non-conserved amino acids. The hot-spots that code for buried or conserved amino acids within the CDRs are preferably not mutagenized. These residues are usually critical for the overall structure and are unlikely to interact with the antigen since they are buried.

Methods of carrying out the above described manipulation of amino acids and protein domains are well known to a person skilled in the art. For example, said manipulations could conveniently be carried out by genetic engineering at the nucleic acid level wherein nucleic acid molecules encoding appropriate binding proteins and domains thereof are modified such that the amino acid sequence of the resulting expressed protein is in turn modified in the appropriate way.

Testing the ability of one or more new binding proteins (or indeed any binding protein) to specifically bind to CD98hc can be carried out by any appropriate method which are well known and described in the art. CD98hc is commercially available (see the Examples) and this can readily be used to assay binding, for example by conventional methods such as ELISA, affinity chromatography, immunoprecipitation, Western blot, etc.

The new binding proteins produced by these methods will preferably have a higher or enhanced affinity (or at least an equivalent affinity) for CD98hc as the parent binding protein and can be treated and used in the same way as the binding proteins of the invention as described elsewhere herein (e.g. for therapy, diagnosis, in compositions etc).

New binding proteins produced, obtained or obtainable by these methods form a yet further aspect of the invention.

Other features and advantages of the present invention will become apparent from the above detailed description. It should be understood, however, that the above detailed description and the following specific examples, while indicating preferred embodiments of the invention, are given by way of illustration only, since various changes and modifications within the spirit and scope of the invention will become apparent to those skilled in the art from the detailed description.

The invention will now be described in more detail in the following non-limited examples with reference to the drawings in which:

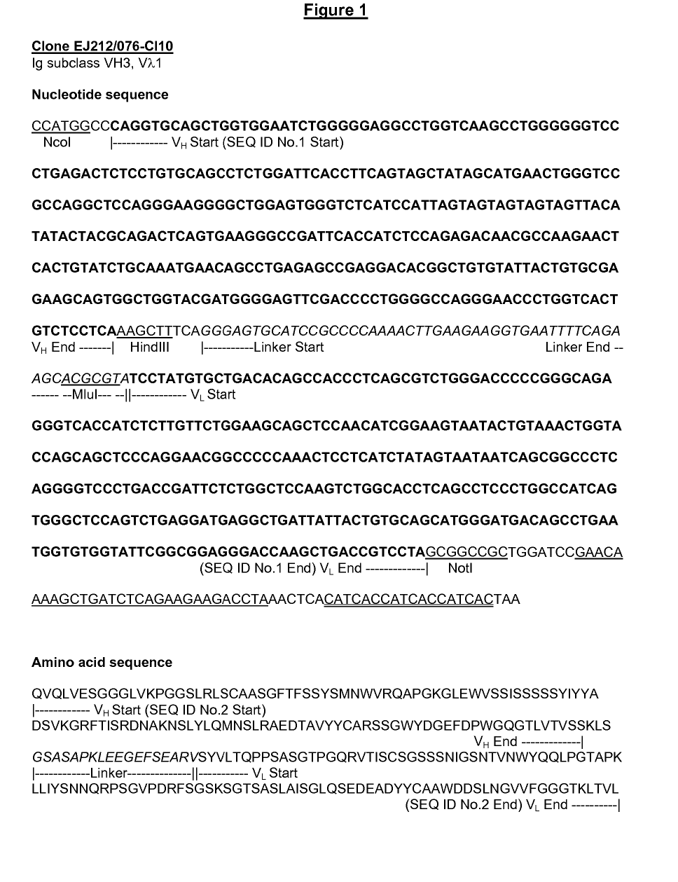

FIG. 1 shows the nucleotide and amino acid sequence of the heavy and light chain of clone EJ121/076-CI10. ScFv were cloned via Nco/NotI site into pHOG21 (3.7 Kb). The restriction sites used for initial cloning (NcoI, HindIII, MluI and NotI) are italicized and underlined. The linker sequence between VH and VL is in italic. The c-myc epitope and 6 His are underlined and double underlined, respectively.

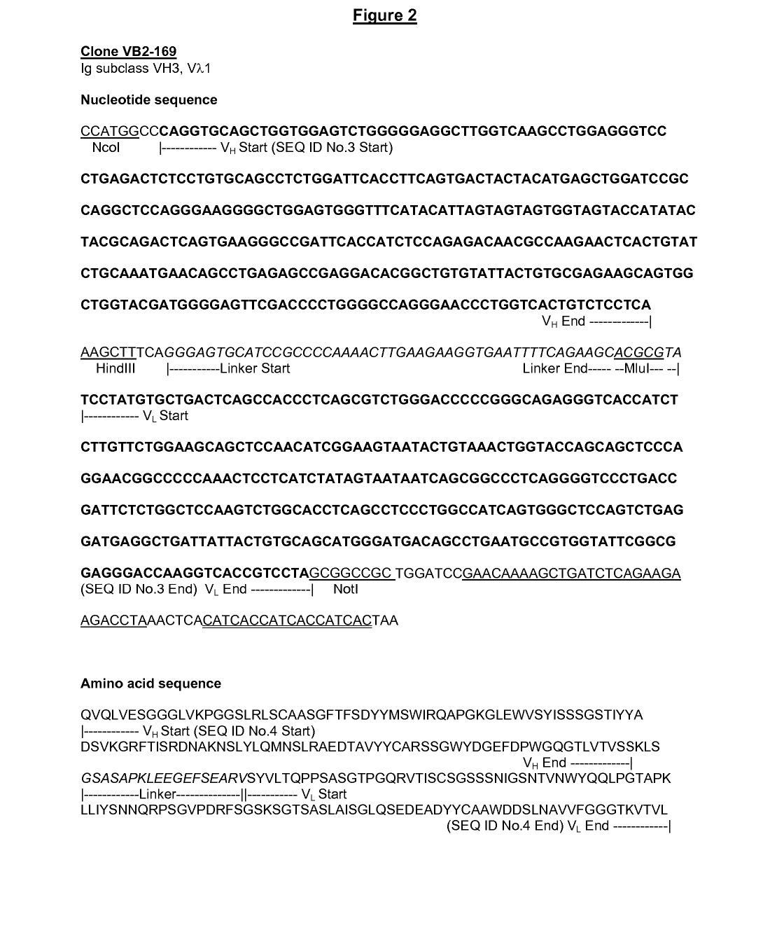

FIG. 2 shows the nucleotide and amino acid sequence of the heavy and light chain of clone VB2-169. ScFv were cloned via Nco/NotI site into pHOG21 (3.7 Kb). The restriction sites used for initial cloning (NcoI, HindIII, MluI and NotI) are italicized and underlined. The linker sequence between VH and VL is in italic. The c-myc epitope and 6 His are underlined and double underlined, respectively

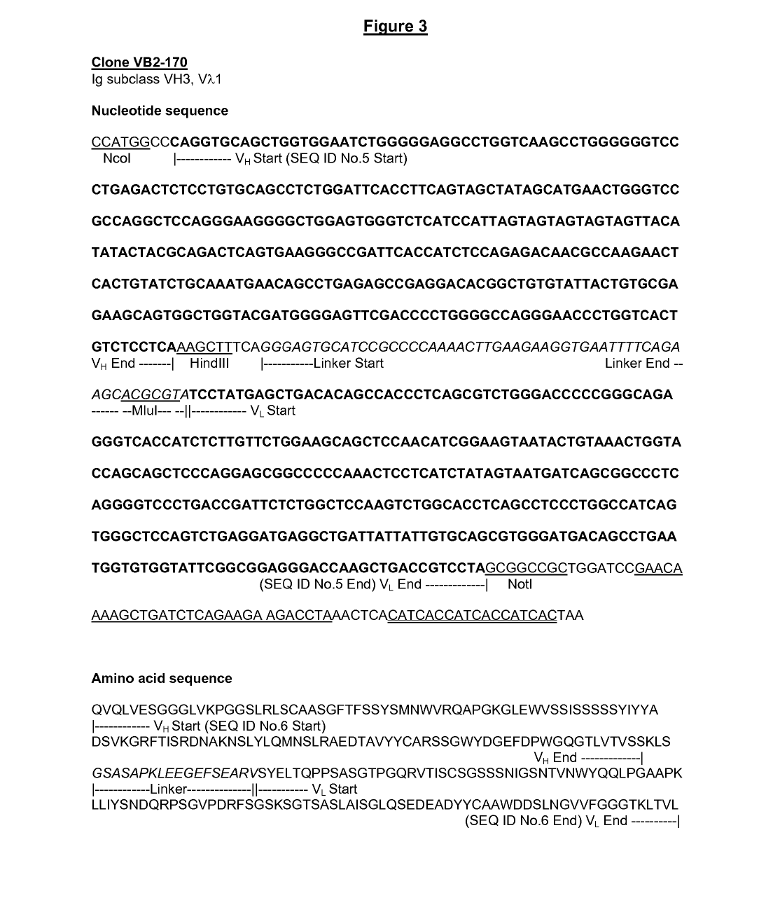

FIG. 3 is the nucleotide and amino acid sequence of the heavy and light chain of clone VB2-170. ScFv were cloned via Nco/NotI site into pHOG21 (3.7 Kb). The restriction sites used for initial cloning (NcoI, HindIII, MluI and NotI) are italicized and underlined. The linker sequence between VH and VL is in italic. The c-myc epitope and 6His are underline and double underline, respectively.

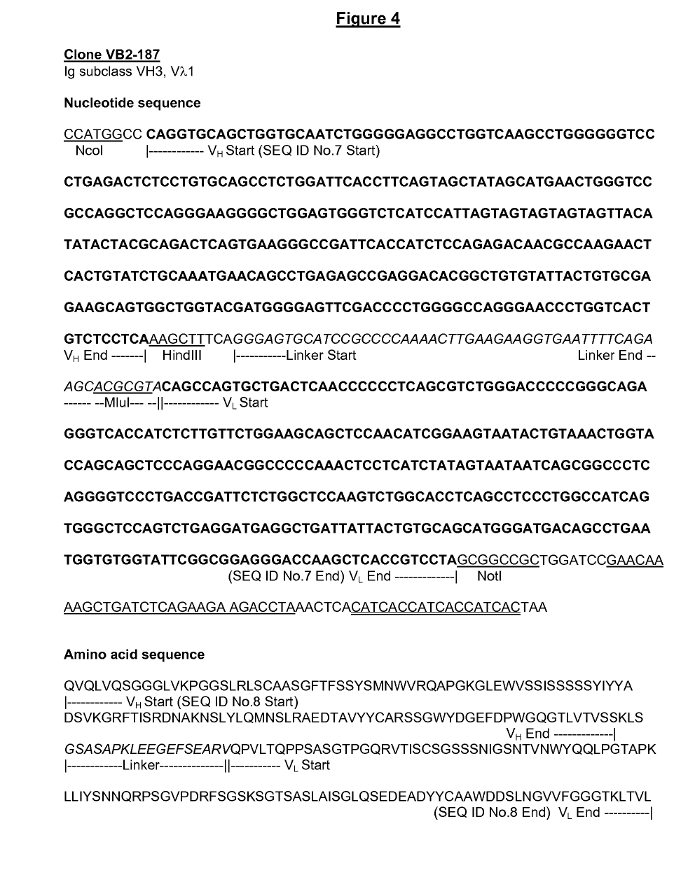

FIG. 4 is the nucleotide and amino acid sequence of the heavy and light chain of clone VB2-187. ScFv were cloned via Nco/NotI site into pHOG21 (3.7 Kb). The restriction sites used for initial cloning (NcoI, HindIII, MluI and NotI) are italicized and underlined. The linker sequence between VH and VL is in italic. The c-myc epitope and 6His are underline and double underline, respectively.

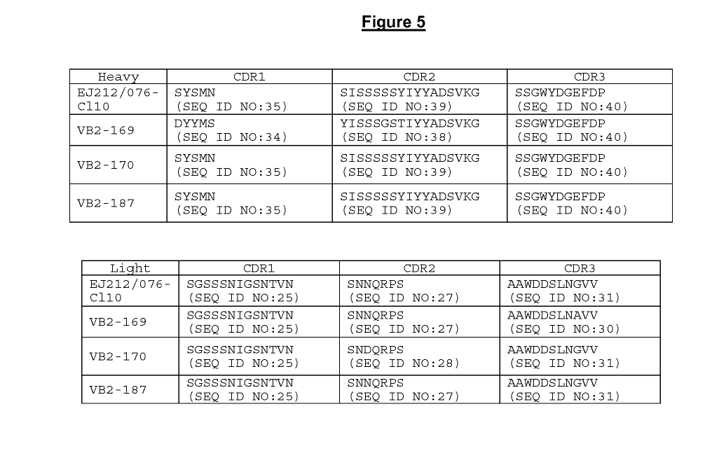

FIG. 5 shows the amino acid sequences of CDR1, CDR2, and CDR3 of the heavy and light chains of clones EJ212/076-CI10, VB2-169, VB2-170 and VB2-187.

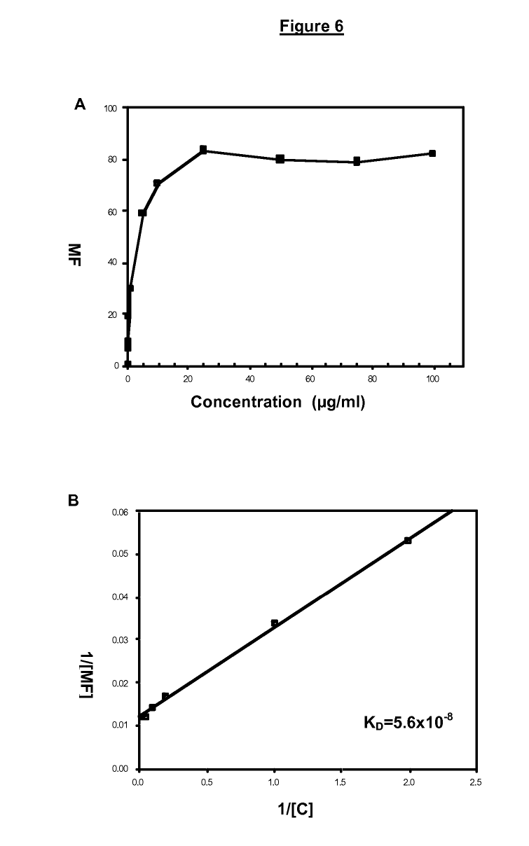

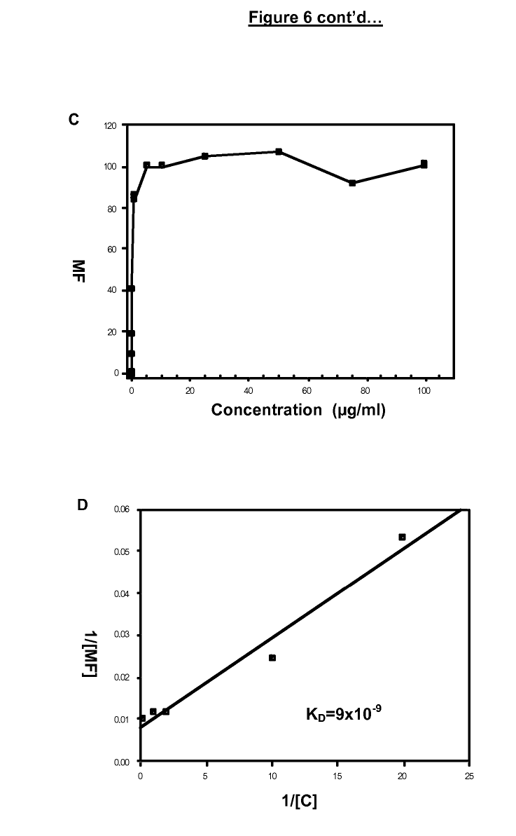

FIG. 6 shows the binding affinity of VB2-169 and VB2-170. A-549 cells were incubated with various concentrations of (A) VB2-169 and (C) VB2-170 and the medium fluorescence was obtained by flow cytometry. The affinity of (B) VB2-169 and (D) VB2-170 was calculated using the Lineweaver-Burk method.

FIG. 7 is an assessment of VB2-169 (Top) and VB2-170 (Bottom) internalization used at 5 μg/mL by confocal microscopy. A-549 cells were incubated with both scFvs for 60 minutes at 4° C., washed and warmed to 37° C. for 60 min. Cell samples before and after incubation at 37° C. were fixed, permeabilized and labeled with mouse monoclonal antibody anti-His tag followed by an FITC-labeled anti-mouse IgG. Fluorescent labeling of A-549 cells after incubation with VB2-169 and VB2-170 at 4° C. for 60 min displayed membrane staining, (60X ×3 magnification) (Left panel). Following incubation of antibody-bound cells at 37° C. for 60 min the cells show strong intracellular staining indicating the internalization of the antibodies, (60X ×3 magnification) (Right panel).

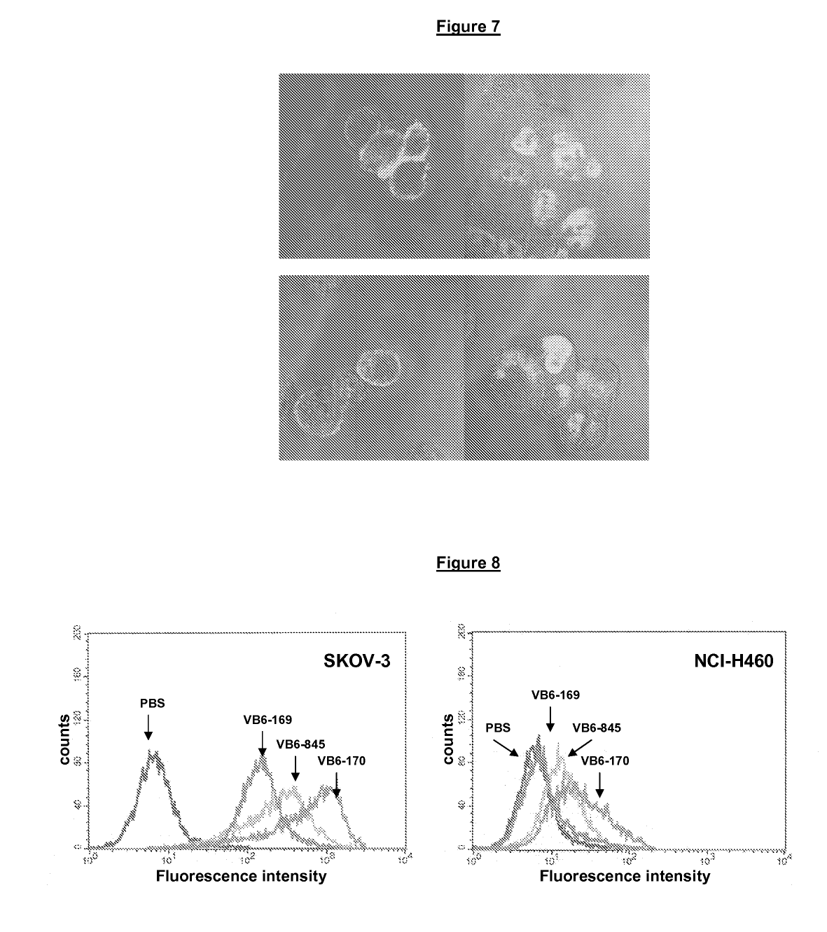

FIG. 8 shows the biological activity of VB6-169 and VB6-170 as measured by flow cytometry and compared to VB6-845. The reactivity and specificity of VB6-169 and VB6-170 was assessed with positive cell line SKOV-3 and negative cell line NCI-H460. A shift in median fluorescence with positive cell line, SKOV-3 was observed after incubation with VB6-169, VB6-170 and VB6-845. In contrast, a marginal shift was observed with PBS as well as with the negative cell line, NCI-H460.

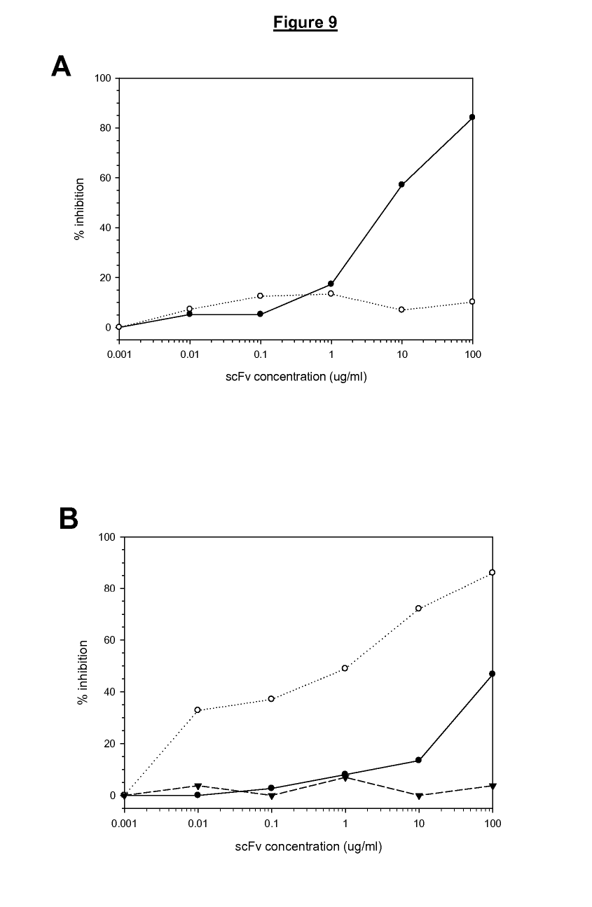

FIG. 9 shows the results of a competition assay. (A) Competition assay using VB6-170 and VB2-170 with A-549 cells. A-549 cells were incubated with a fixed concentration of VB6-170 (10 μg/mL) and increasing concentrations of VB2-170 (black circle) and 4B5 (open circle) ranging from 0 to 100 μg/mL. The percentage of inhibition was calculated using the following formula: % of inhibition=[1−(medium fluorescence in presence of VB2-170/medium fluorescence in absence of VB2-170)]×100. (B) Competition assay using VB6-169 and VB2-169, VB2-170 and 4B5 scFv with A-549 cells. A-549 cells were incubated with a fixed concentration of VB6-169 (10 μg/mL) and increasing concentrations of VB2-169 (black circle), VB2-170 (open circle), and 4B5 (black triangle) ranging from 0 to 100 μg/mL.

FIG. 10 is representative photographs of immunohistochemical staining of normal heart (upper left panel), normal lung (upper right panel), normal liver (lower left) and normal brain tissues (lower right) with VB6-170 used at 5 μg/mL. 400× magnification. Arrow indicates membrane staining.

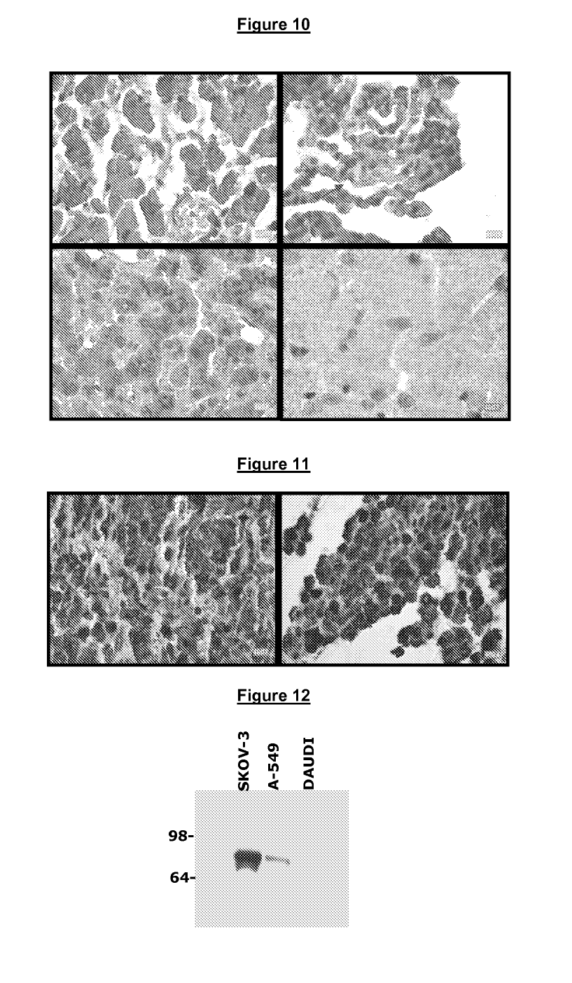

FIG. 11 is representative photographs of immunohistochemical staining of human breast tumor tissue (left panel) and prostate tumor tissue (right panel) with VB6-170 used at 5 μg/mL. 400× magnification.

FIG. 12 is a 1D PAGE/Western analysis of antigens purified from SKOV-3, A-549 and Daudi. 1 mg equivalents of membranes were used to immunopurify the antigen binding to VB6-170. No bands were seen when the same blot was probed with VB6-4B5.

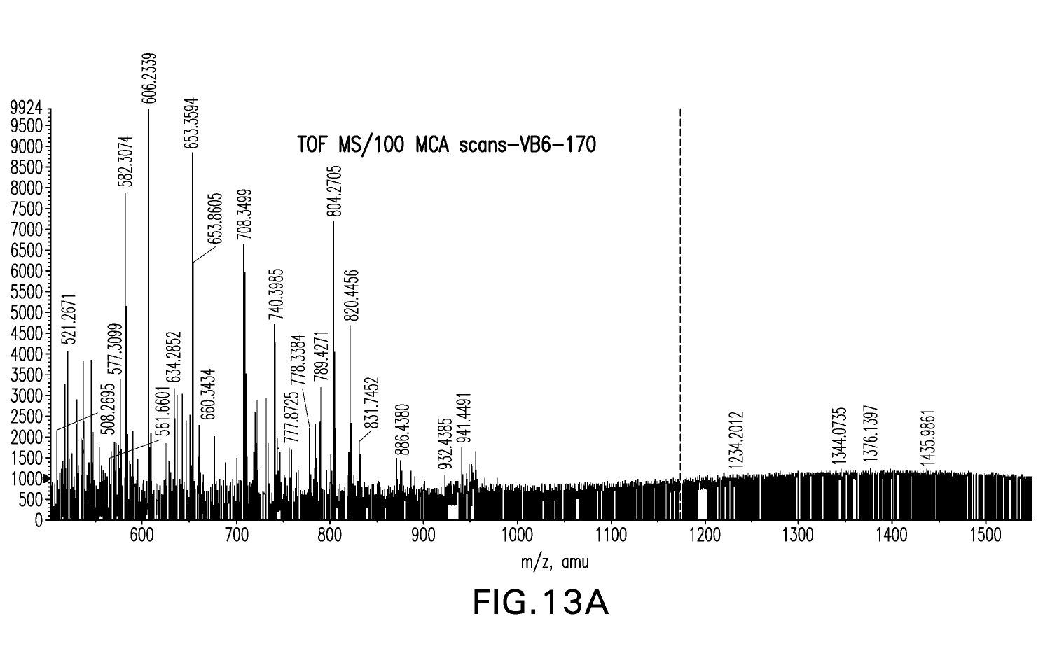

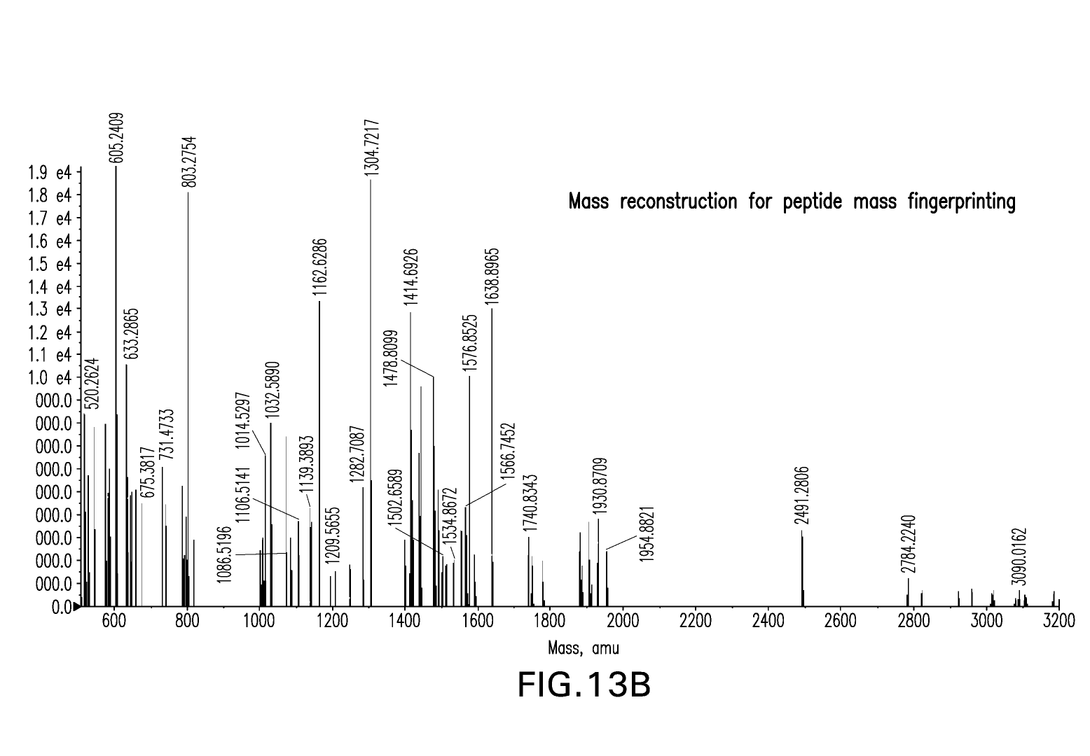

FIG. 13 is a TOF-MS (survey scan) to detect the presence of all the peptide ions in the sample. Hundred scans at 900-1400V in the range of 100-1550 amu on a static nanospray installed on a QSTAR-pulsar-i (ESI-qTOF-MS/MS) system resulted in the recovery of a significant number of peptides, which when analyzed yielded the protein ID as CD98hc/SLC-3A2 gene product.

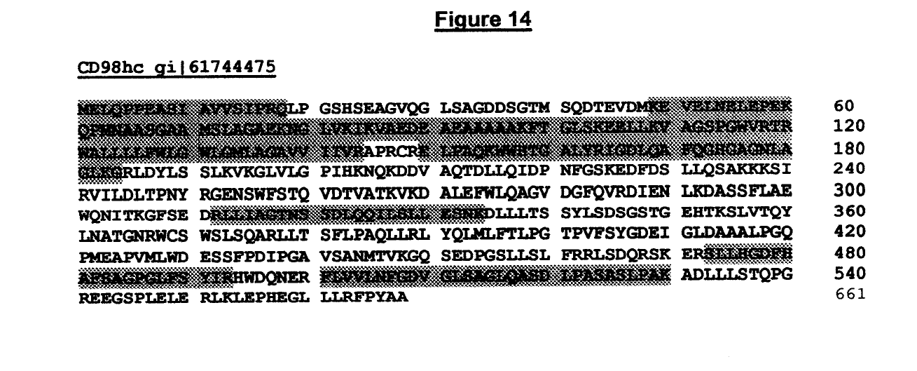

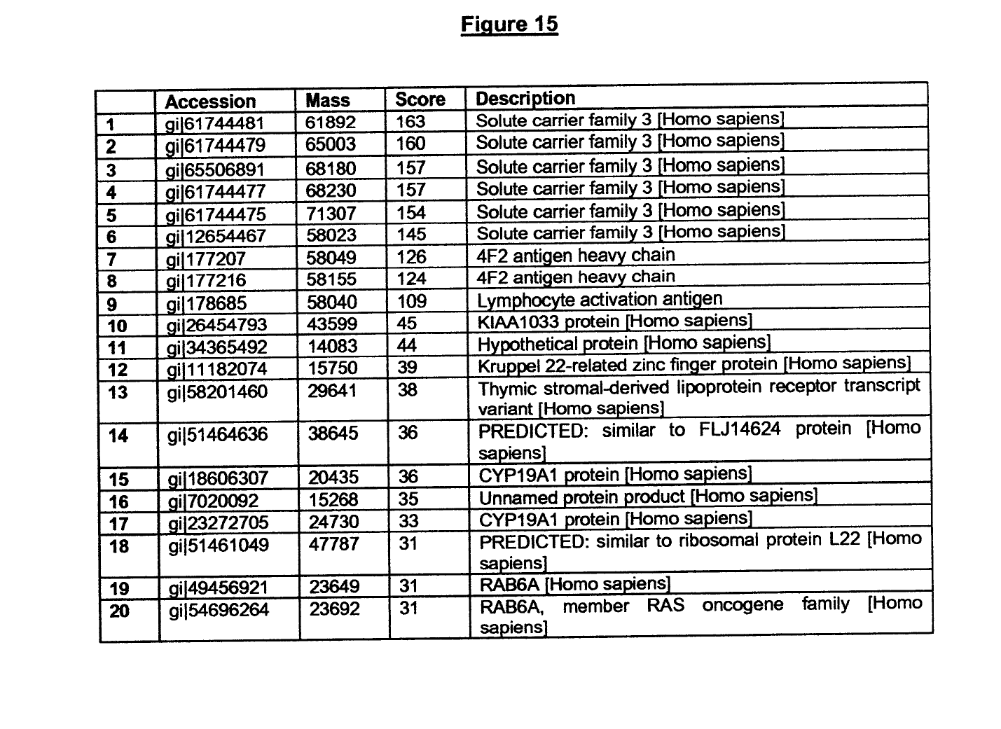

FIG. 14 shows the sequence coverage of the peptides of Table 8 in respect to the CD98hc sequence (Table 8 shows the sequences of 15 peptides recovered from in-gel tryptic digestion in-house and analysed by mass spectrometry). All of the peptides show 100% homology to the CD98hc sequence in the database and provided 32.6% coverage of the protein.

FIG. 15 shows the results of the peptide sequence homology search in the protein database. The identified antigen, CD98hc has a highly significant score of 163. Due to the nature of the database server and the similarity/homology linked proteins, all the isoforms of this protein were pulled down as hits. However, MS/MS fragmentation and identity of peptides clarifies that the antigen is isoform α2, otherwise referred to as CD98hc or 4F2 antigen.

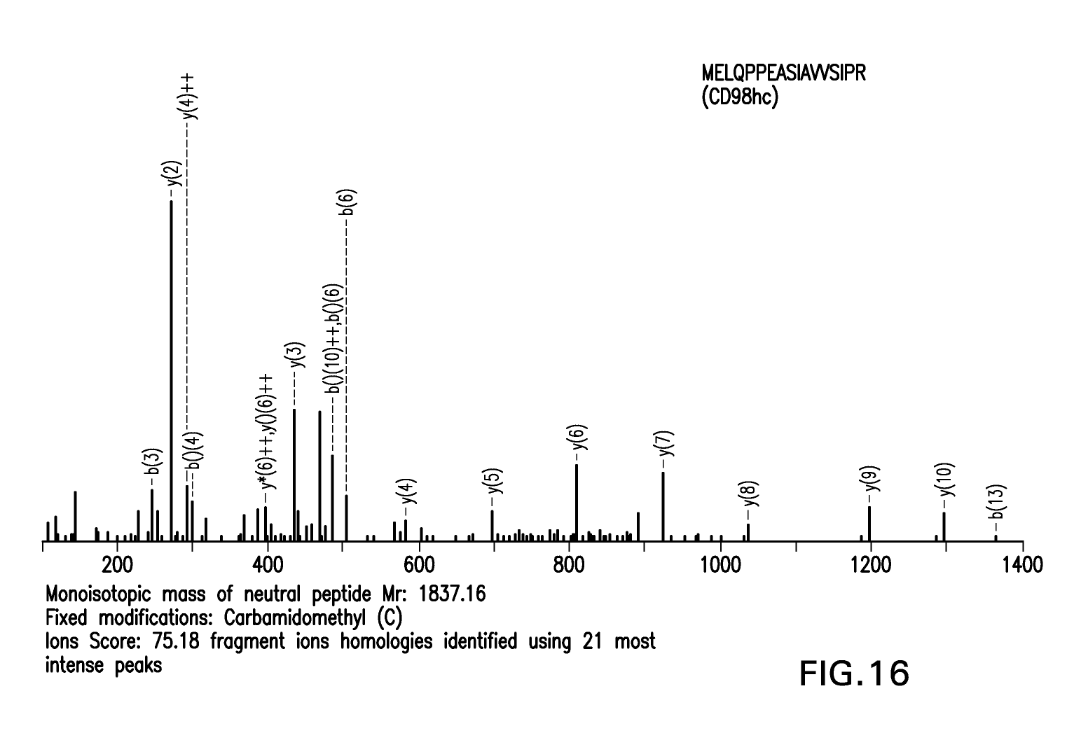

FIG. 16 shows the MS/MS ion fragmentation of the neutral peptide Mr. 1837.16, appearing as a doubly charged molecule (908.0002+). The peptide sequence exactly matched the peptide from CD98hc

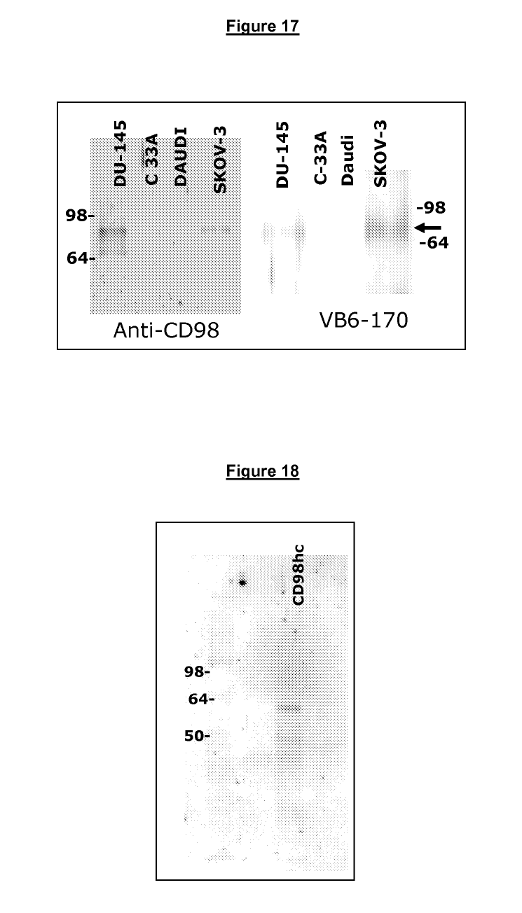

FIG. 17 shows the immunoprecipitates from SKOV-3, DU-145, C-33A and Daudi, which were separated by electrophoresis and transferred by Western blotting. Blots were then probed with both anti-CD98 and VB6-170. Arrows indicate the position of the proteins detected by chemiluminescence.

FIG. 18 shows the results of a validation experiment. Commercially available CD98hc from Santa Cruz biotechnology Inc., (Cat # SC-4371 WB), expressed as a 60 kDa fusion tagged protein in E. coli, showed positive reactivity to VB6-170 on western blot analysis.

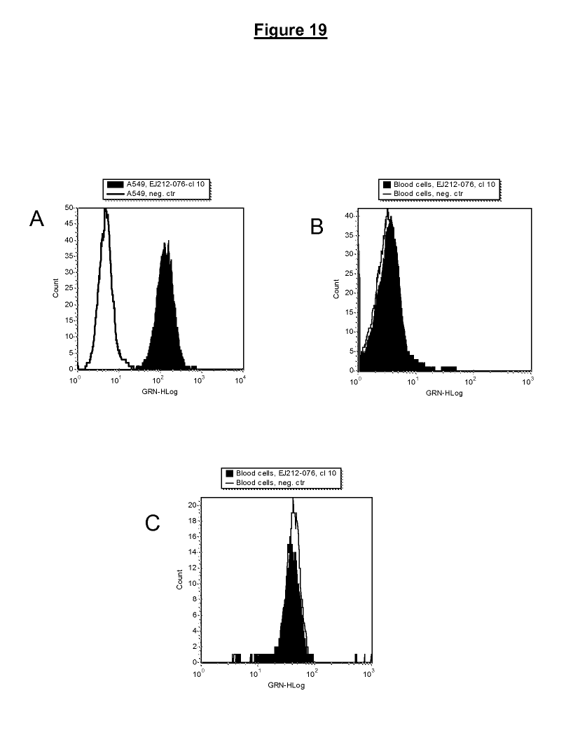

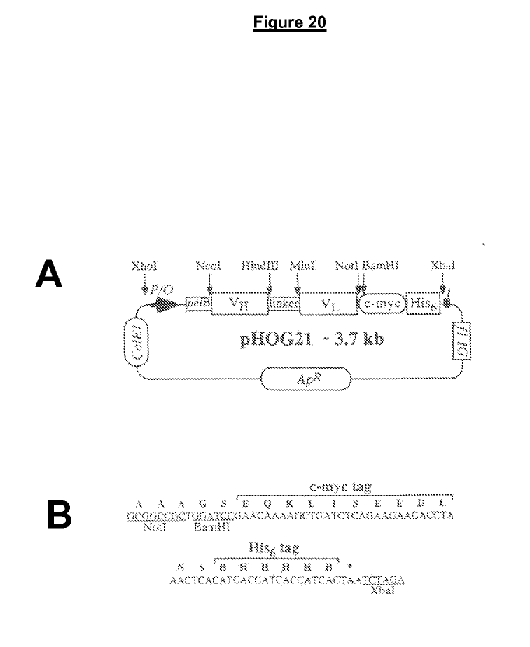

FIG. 19 shows binding of EJ212/076-CI10 to A-549 (A), PBLs (B) and granulocytes (C) by Guava EasyCyte measurements. A clear shift in mean fluorescence was seen with A-549 after incubation with EJ212/076-CI10 (black shading) as compared with a negative control antibody (white shading). No binding of EJ212/076-CI10 over the negative control antibody was observed with PBLs and granulocytes.

FIG. 20A shows the scFv expression vector pHOG21. ApR, Ampicillin resistance gene; ColE1, origin of DNA replication; flIG, intergenic region of phage f1; c-myc, epitope recognized by the monoclonal antibody 9E10; His6, six histine residues; pelB, signal peptide of bacterial pectate lyase; P/O, wild type lac promoter operator. FIG. 20B shows the nucleotide and amino acids sequences of the C-terminal coding region.

The following non-limiting examples are illustrative of the present invention:

### EXAMPLES

### Example 1

### Novel Antibodies

Given the need for tumor specific antibodies, human antibodies have been identified which are reactive against A-549 lung tumor cells and negative against PBL cells. Single chain forms of antibodies were cloned in the pHOG21 plasmid (at the NcoI and Not I restriction sites) which contains a c-myc and 6×His tag epitopes (Kipriyanov, et al. High level production of soluble single chain antibodies in small-scale Escherichia coli cultures. J. Immunol. Methods 200(1997), p69-77) E. coli cells, XL-1 blue, were transformed, selected on ampicillin plates and the scFv was expressed upon IPTG induction. Purified scFv were tested by ELISA for selective biological activity with A-549 versus PBL cells. The biological activity was further confirmed by flow cytometry.

Sequencing

The nucleotide sequences of the heavy and light chains of four antibody producing clones were sequenced. The antibodies are designated as EJ212/076-CI10, VB2-169, VB2-170 and VB2-187. The nucleotide sequence and amino acid sequence of the light and heavy chains of EJ212/076-CI10, VB2-169, VB2-170 and VB2-187 are shown in FIGS. 1, 2, 3 and 4, respectively. The CDR regions of the light and heavy chains of EJ212/076-CI10, VB2-169, VB2-170 and VB2-187 are shown in FIG. 5.

### Example 2

### Specificity and Selectivity of VB2-169 and VB2-170

The tumor cell line panels used in this study are summarized in Table 1. Tumor cell lines were maintained in culture following the procedures provided by ATCC. Cancer cells were harvested following the standard procedures.

VB2-169 and VB2-170 were tested by flow cytometry to determine their pattern of tumor cell reactivity. Briefly, cell suspensions at 0.3×10⁶ cells/300 μL were treated with 1 μg/mL of purified scFv or controls which consisted of 1) PBS-5% FCS, 2) an isotype-matched antibody 4B5 scFv (negative control) and 3) an anti-EpCAM scFv immunoconjugate (positive control). Bound scFvs were detected with an anti-His tag mouse monoclonal antibody (Amersham) followed by a FITC-labeled anti-mouse IgG. The assay was repeated twice and the results were expressed as the mean of the fold-increase in median fluorescence over the isotype-matched control in two experiments.

The results for all cell lines are summarized in Table 1. VB2-169 and VB2-170 bound to all tumor cell lines with 2 to 8-fold and 7 to 30-fold increases, respectively. The strongest binding was observed with, but not limited to endometrial, ovarian and prostate cancer cell lines.

### Example 3

### Binding Affinity of VB2-169 and VB2-170

Flow cytometry was used to assess the binding affinity of both antibodies. Increased concentrations of the scFvs were tested against a fixed number of A-549 cells, a human lung cell line, to establish a saturation curve. Bound scFv was detected as described above. The binding affinity expressed as the dissociation constant, K_{D} was calculated by the Lineweaver-Burk method by plotting the inverse of the median fluorescence as a function of the inverse of the antibody concentration. The dissociation constant was determined by the following equation: 1/F=1/F_{Max}+(K_{D}/F_{Max})(1/[scFv]), where F corresponds to the background subtracted median fluorescence and F_{Max} was calculated from the plot.

The binding curves are shown in FIG. 6 and the K_{D} value were calculated as 5.6×10⁻⁸ M and 9×10⁻⁹ M for VB2-169 and VB2-170, respectively.

### Example 4

### Assessment of VB2-169 and VB2-170 Internalization by Flow Cytometry and Confocal Microscopy

Internalization of VB2-169 and VB2-170 was assessed by temperature-differential fluorescence labeling on flow cytometry. Detection of bound scFv is described above. H11 scFv known to be an internalizing scFv was used as the positive control. Percent reduction in median fluorescence in response to VB2-169 or VB2-170 treatment of A-549 cells for 60 or 120 min at 37° C. was calculated. To confirm the temperature-dependent uptake of VB2-169 and VB2-170, scFv-treated cells were further analyzed for their membrane and intracellular staining by confocal microscopy.

Table 2 shows representative flow cytometry data. After 60 min at 37° C., the membrane-bound VB2-169 and VB2-170 disappeared from the cell surface, with a reduction of 77% and 84.5%, respectively, in median fluorescence. Increasing the incubation time at 37° C. was associated with a further decline in median fluorescence. By 120 min, the median fluorescence had decreased by 80.8 and 88.1%, respectively.

To confirm that the cell-surface bound VB2-169 and VB2-170 were internalized into A-549 rather than shed from the membrane, scFv-treated cells were further evaluated for their membrane and intracellular staining by confocal microscopy. FIG. 7 illustrates the results. Like H11 scFv (an antibody which is known to internalize and used as a positive control), the incubation of A-549 cells with VB2-169 (Top) and VB2-170 (Bottom) at 4° C. (FIG. 7, left panel) demonstrated a circumferential surface distribution of the fluorescence label. Warming the scFv-bound cells to 37° C. resulted in the internalization of both the scFvs as revealed by the punctuate pattern of intracellular staining (FIG. 7, right panel).

### Example 5

### Detection of VB6-170 and VB6-169 Binding by Flow Cytometry

Further Studies Using Fab-Cytotoxin Fusion Protein Format

Because these antibodies internalize, one potential utility is in the development of immunoconjugates to carry medically relevant agents inside cells. To determine whether the 169 and 170 antibodies were suitable for this application, they were also engineered using the Fab format for the antibody portion fused a cytotoxic protein. In this format the antibodies are referred to as VB6-169 and 170.

Human lung (A-549 and NCI-H460) and human ovarian (SKOV-3) cell lines were grown in their respective media as per ATCC protocols. Cells were harvested at 30 to 40% confluency with viability greater than 90%. NCI-H460 was used as negative cell lines. VB6-845 (anti-Epcam) was used as positive control. Briefly, VB6-170, VB6-169 and VB6-845 were incubated with 0.45×10⁶ tumor cells for 1.5 hours on ice. After washing, cells were incubated with rabbit anti-cytotoxin (1/100) for an hour on ice. The cells were washed and incubated with FITC-conjugated anti-rabbit IgG for an additional 30 minutes on ice. Subsequently, the cells were washed, resuspended in PBS 5% FCS containing propidium iodide for assessment of Fab binding by flow cytometry.

Antibody profiling by measuring tumor cell line reactivity showed that both VB2-169 and VB2-170 bind to the positive cell line SKOV-3 but not to the negative cell line, NCI-H460. As expected, little binding was detected with VB6-169 and VB6-170 with NCI-H460 (FIG. 8). In contrast, a shift in median fluorescence was observed with VB6-169, VB6-170 and VB6-845 incubated with SKOV-3. The shift in median fluorescence of VB6-170 was higher than VB6-169 and is probably linked to their corresponding affinity.

### Example 6

### Competition Assay

The analysis of the CDR regions of VB2-169 and VB2-170 indicated that all residues of the CDR3 loops were identical except one. Furthermore the two antibodies showed a similar cell binding profile. In order to determine whether VB6-169 and VB6-170 recognized to the same cell surface antigen, they were tested in a competition assay.

First, the impact of the new antibody format (Fab) and the fused cytotoxin on cell binding was assessed. A fixed concentration of VB6-170 or VB6-169 was incubated with A-549 in presence of increasing amount of their corresponding scFv ranging from 0 to 100 μg/mL. The bound VB6-170 was detected by flow cytometry using anti-cytotoxin antibody. FIG. 9A shows that the increased concentrations of the VB2-170 result in a decrease of the VB6-170 binding suggesting that the specificity of the engineered Fab was preserved. Similar concentrations of 4B5, a non binding scFv, have no inhibitory effect on the binding activity of VB6-170 demonstrating the specificity of the competition.

Second, bound VB6-169 to A-549 cells was measured in presence of increased concentration of VB2-170 (FIG. 9B). The analysis of the data clearly showed that VB2-170 inhibits the binding of VB6-169 suggesting that they compete for the same antigen. The higher affinity of VB2-170 could explain the greater inhibition compared to VB2-169. Since the two antibodies appear to recognized the same antigen but the 170 has a slightly higher affinity, subsequent experiments, including antigen identification studies, were conducted mostly with the 170 antibody. A third antibody (VB2-187) was also obtained and found to share nearly 100% identity in its CDR with the 170 antibody and to compete with it. It is therefore expected that it binds to the same antigen as 169 and 170.

### Example 7

### Selectivity and Specificity of VB6-170 on Tissue Microarrays

The selectivity of VB6-170 was evaluated by determining the binding to critical normal tissues from human donors using immunohistochemistry staining. An absence of cell membrane binding to these critical normal tissues is an important characteristic for an antibody to be developed as a cancer therapeutic since it suggest that the antibody will not bind and target normal tissues in vivo. This information is then coupled to that from the binding studies to a tumor tissue microarray to evaluate the clinical potential of the antibody.

VB6-170 was first tested against fixed A-549 cell line pellets to define the optimal conditions for staining.

The antibody was then tested on a low-density array of critical normal tissues including brain, colon, heart, kidney, liver, lung, pancreas and stomach from 5 different donors, using 2 cores per donor. VB6-170 showed no membrane staining except for the lung where a weak staining was observed on the membrane (1+ with 10-50% of positive cells) and believed to be non-specific and due to an edge effect (FIG. 10, Top right and Table 3). However, cytoplasmic staining with an overall score of 1-2 and a percent of positive cells varying from 10 to 100% was observed for all tissues (FIG. 10).

VB6-170 at the same concentration (5 μg/mL) was used to stain a high-density array of tumor tissues including breast, colon, prostate, kidney, liver, lung, ovary, pancreas, head & neck, and skin from 9 different donors, using 2 cores per donor. The results are summarized in Table 4. All but the kidney showed membrane staining. The strongest binding was observed with, but not limited to breast, lung and prostate with a highest score at 2+ and an overall percent of positive cells greater than 50%. In all cases, staining of the cytoplasm with 1+ score was observed. FIG. 11 showed examples of the staining to representative tissues.

### Example 8

### Characterization of the VB6-170 Antigen

Method

VB6-170 and VB6-4B5 (isotype-matched control) antibodies were equilibrated with 0.9M sodium borate buffer, pH 9.5 and made to bind to rabbit anti-cytotoxin at 2-8° C. for 16 hours. Unbound excesses of the anti-cytotoxin antibody were removed subsequently by centrifugation at 4000 RPM for 10 minutes. Protein-G-sepharose beads were then added and the VB6-170NB6-4B5 mixtures were nutated at room temperature for 2 hours.

Equal amounts of membrane preparations from 3 positive (SKOV-3; DU-145; A549) and negative cell lines, (C33A and DAUDI) were nutated with 40 μL of immobilized beads representing 20 μg of VB6-170NB6-4B5, in the presence of protease inhibitors with conditions mimicking in vivo conditions. Immune complexes were centrifuged, washed with RIP-A lysis buffer and eluted with 0.2 M glycine pH 2.5. Immunoprecipitations were carried out on two very positive cell lines, (i.e., SKOV-3 and DU-145), one moderately positive cell line, (A-549), and two weakly positive cell lines, i.e., MDA-MB231 and PC-3. Two negative cell lines, (C-33A and Daudi) with VB6-170 and equal amounts of VB6-4B5 were processed in parallel each time.

The purified proteins were subjected to reducing and non-reducing conditions of sample preparation and were subsequently analyzed by SDS-PAGE/Western Blotting. The resulting blots were probed with the required antibodies and corresponding secondary antibodies conjugated to HRP, to visualize the immuno-purified proteins by chemiluminescence.

Proteins excised from the 1D-gel were digested with sequencing grade trypsin in a 20-hour peptide extraction process and the extracted peptides analyzed on a QSTAR Pulsar-I (ESI-qTOF-MS/MS).

Peptide masses extracted from the mass spectra were used directly to identify the antigen according to the MOWSE scores obtained on protein databases that are accessible through search engines such as MASCOT, SEQUEST, and Prospector. De-novo sequencing of the identified proteins was also performed. Peptides were extracted from both positive and negative cell lines

Results

Membrane proteins from SKOV-3 and DAUDI were subjected to SDS-PAGE and Western blotting. One band was down-regulated or absent in DAUDI at ˜70±5 kDa regions, suggesting that the antigen had MW of about 70 kDa. Flow results before and after enzyme treatments indicated that the epitope reactive to VB6-170 was not glycosylated.

1D-PAGE/Western Analysis

A single band was detected after separation on a 1D-PAGE at ˜70 kDa under reducing conditions (FIG. 12) in antigen-positive cell lines (SKOV-3, A-549). The same band was absent in the negative cell line (DAUDI). One mg equivalent of membrane protein was used for the purification process. None of the cell lines showed positive immunoprecipitation with VB6-4B5. The Western data is summarized in Table 5. The differentially expressed band at ˜70 kDa was excised from both SKOV-3 and A-549 and used for peptide extractions subsequently leading to the protein ID. Two other weakly binding cell lines, PC-3 and MDA-MB-231 showed the same antigen expression pattern as seen with SKOV-3 and A-549.

C-33A were screened under non-reducing conditions. Immunoprecipitations were as described earlier. a ˜70 kDa band was present in all the positive cell lines, but absent in C-33A.

Peptide Extraction and Protein Analysis

SKOV-3 and A-549 membranes were used to immunopurify antigen(s) that bind specifically to VB6-170. A ˜70 kDa band was observed in both the cell lines, but was absent in Daudi. A gel slice corresponding to the 70 kDa band was excised along with a corresponding gel slice from the C-33A and processed for peptide extractions. Results of the analysis on a QSTAR Pulsar-I (ESI-qTOF-MS/MS) are shown in FIG. 13.

Analysis of Peptide Masses and Their Identities

Peptide analysis was done in two ways:
All the peptides recovered and reconstructed to their right masses were used directly in a peptide mass fingerprinting step to obtain an ID for the protein. The list of peptides recovered and their sequence are shown in Table 8. All peptides represented were obtained by denovo sequencing. FIG. 14 shows their mapped positions in respect to the sequence of CD98hc.
Peptides that were abundant and well ionized were chosen for further MS/MS ion fragmentation, wherein, the ‘y’ and ‘b’ ions were used to deduce their primary structure. These sequences were then searched for homologies in the protein database for protein ID (FIG. 15). All of the peptides show 100% homology to the CD98hc sequence in the database.
   MS/MS Fragmentation of Peptide 1837.16

MS/MS fragmentation of one of the peptides (1837.16−908.000 2+) gave rise to the fragment ions shown below (FIG. 16), that mapped to a peptide from CD98hc/SLC-3A2 gene product.

Since peptide 1837.16 (908.000 2+) was the most abundant peptide detected in TOF-MS, this peptide was used for MS/MS ion fragmentation apart from the peptides derived from mass fingerprinting. The peptide derived from the spectra clearly matched the sequence on CD98hc, and therefore was pulled down as a major hit. This ion fragmentation data further confirms the identity of CD98hc as the cognate antigen for VB6-170.

Validation of the Antigen Identified

Since the antigen identified was well-known and commercially available, recombinant antigen and a corresponding antibody (anti-CD98hc) were purchased and probed with VB6-170 and used as a probe for VB6-170 purified antigens, respectively, (FIGS. 17 and 18).

An immunoprecipitation experiment was set up with VB6-170 and the purified proteins were separated on SDS-PAGE and transferred to nitrocellulose membranes to be probed by anti-CD98 and VB6-170. As can be seen from FIG. 17, both the antibodies detected the presence of the same protein, indicating a high level of homology between them.

Since it has been established that CD98hc is the antigen that binds cognately to VB6-170, another experiment was designed using the recombinant CD98hc. The recombinant CD98 protein, (commercially available as a 60 kDa fusion protein), was resolved, transferred and probed with VB6-170. As can be seen from FIG. 18, a single band at 60 kDa was detected with VB6-170.

The predicted 60 kDa fusion protein is detected with VB6-170, confirming specificity in the identification process.

### Example 9

### Specificity of EJ212/076-CI10 by Guava EasyCyte

To determine the tumor-specificity of the antibody, binding of EJ212/076-CI10 was tested by Guava EasyCyte (Guava Technologies) on A-549, PBLs and granulocytes. Briefly, 1.2×10⁵ cells/100 μl were incubated with 10 μg/ml purified EJ212/076-CI10 or PBS. Bound scFv were detected with an anti-c-myc tag mouse monoclonal antibody (Invitrogen) followed by a FITC-labeled anti-mouse IgG (DAKO).

Results showed strong binding of EJ212/076-CI10 to A-549 (FIG. 19 A), but no or insignificant binding to PBL or granulocytes (FIG. 19 B and C, respectively).

Conclusion

VB2-169 and VB2-170 (scFv format) were shown by flow cytometry to bind to various cancer cell lines and to have a binding affinity of 10⁻⁸ to 10⁻⁹M. Both antibodies are internalized into the A-549 cells as demonstrated by confocal microscopy. Competition tests performed between VB6-169 and VB6-170 (Fab-cytotoxin format) confirm that these two antibodies are directed to the same antigen. Therefore, only the 170 antibody was further tested. The Fab version of the antibody, VB6-170, shows no significant reactivity to normal tissue tested and excellent reactivity to tumor tissue microarrays with stronger binding to the breast, prostate and lung.

CD98hc was identified as the antigen for VB6-170 (and, by extension, for VB6-169, VB2-187 and EJ212/076-CI10) using SDS/Page separation, digestion and mass spectrometry analysis. SDS-PAGE/Western blot analysis of recombinant form of CD98hc (expressed and commercially available as a 60 kDa fusion tagged protein) probed with VB6-170 showed positive reactivity in the predicted MW range as one strong single band. Immunoprecipitation studies with VB6-170 and subsequent Western blot analysis with VB6-170 and with anti-CD98, demonstrated positive reactivity to the same molecule in both cases. Anti-CD98 failed to react to both the negative cell lines tested. These results indicate that CD98hc is the cell surface antigen that is recognized by VB6-170.

EJ212/076-CI10 (scFv format) was shown by flow cytometry to be tumor specific in that it showed high levels of binding to A549 tumor cells but no or insignificant binding to PBLs or granulocytes.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Comparison of tumor cell surface binding among different clinical indications

| Clinical | Representative | | VB2-169 | |
|---|---|---|---|---|
| indication | Tumor cell lines | N¹ | MF² | VB2-170 MF² |
| Endometrial | RL-95-2, HEC-1-A | 2 | 8.60 ± 0.42 | 30.10 ± 7.53 |
| Ovarian | NIH-OVCAR3, SKOV-3, TOV-112D | 3 | 4.58 ± 2.05 | 22.61 ± 15.96 |
| Prostate | DU-145, PC-3, LNCaP | 3 | 4.36 ± 1.83 | 21.37 ± 12.83 |
| Pancreas | PANC-1, BxPC-3, MIA PaCa-2 | 3 | 3.68 ± 0.74 | 13.69 ± 5.34 |
| Cervix | ME-180, C-4I, C-33A | 3 | 4.29 ± 1.63 | 11.35 ± 5.38 |
| Breast | MDA-MB-435S, MDA-MB231, MCF-7 | 3 | 4.28 ± 1.20 | 10.25 ± 5.04 |
| Lung | A549, NCI-H460, NCI-H69 | 3 | 2.77 ± 1.26 | 9.81 ± 7.99 |
| Colon | HT-29, WiDr, SW-480 | 3 | 3.70 ± 1.88 | 8.50 ± 5.68 |
| Liver | SK-Hep1, HepG2 | 2 | 2.22 ± 1.25 | 7.35 ± 6.40 |
| Stomach | AGS, NCI-N-87, Kato III | 3 | 4.24 ± 0.95 | 6.93 ± 2.11 |

¹Designates the number of cell lines tested per indication.
²Indicates the average of the mean fold increase in median fluorescence over the scFv control from all cell lines in each indication ± SEM.

TABLE 2
Flow cytometry assessment of VB2-169 and VB2-170 binding
to A-549 as a function of time and temperature

| | | Median | Fold- | % |
|---|---|---|---|---|
| | Incubation | Fluorescence | increase | Reduction |
| scFv¹ | time (min) | (MF) | in MF² | in MF³ |
| VB2-169 | 4° C., 120 | 70.41 | 12.6 | |
| 5 μg/mL | 37° C., 60 | 16.4 | 2.9 | 77 |
| | 37° C., 120 | 13.53 | 2.43 | 80.8 |
| VB2-170 | 4° C., 120 | 58.82 | 10.56 | |
| 0.25 μg/mL | 37° C., 60 | 9.14 | 1.64 | 84.5 |
| | 37° C., 120 | 7.04 | 1.26 | 88.1 |
| H11 | 4° C., 120 | 113.42 | 20.36 | |
| 100 μg/mL | 37° C., 60 | 6.67 | 1.2 | 94.1 |
| | 37° C., 120 | 4.57 | 1 | 100 |

¹A representative experiment is shown.
²MF fold-increase above the negative control, 4B5 scFv.
³Percent reduction of MF from the cell-surface of tumor cells.

TABLE 3
LD array of critical normal tissue for VB6-170

| | Membrane | Score | |
|---|---|---|---|
| Tissue | Staining | Range* | Comments |
| Brain | None (0/5) | 0 | 1+ cytoplasmic staining seen in 10-80% of the cells |
| Colon | None (0/5) | 0 | 1+ cytoplasmic staining seen in 10-80% of the cells |
| Heart | None (0/5) | 0 | 1+ cytoplasmic staining seen in 100% of the cells |
| Kidney | None (0/5) | 0 | 2+ cytoplasmic staining in 80%, tubular epithelium (distal) |
| Liver | None (0/5) | 0 | 1-2+ cytoplasmic staining in 20-80%, cytoplasmic granules in one case |
| Lung | None (4/5) | 1+ | Membrane staining is 10-50%, staining is believed to be non-specific and |
| | | | due to an edge effect; 1+ cytoplasmic staining seen in 60-70% of the cells |
| Pancreas | None (0/5) | 0 | 1-2+ cytoplasmic staining in 20-80% of the cells |
| Stomach | None (0/5) | 0 | 1-2+ cytoplasmic staining in mostly 10-20 of the cells with a rare case of |
| | | | 100% |
| *Slides were scored on a 0-3+ scale, with 0 = no staining and trace being less than 1+ but greater than 0. Grades 1+ to 3+ represented increased intensity of staining, with 3+ being strong, dark brown staining. The concentration of VB6-170 used was 5 μg/mL. | | | |
| # For cells staining 1+, the staining was generally, focal with less than 10% of the cells staining. For cells staining 2+, staining was observed in greater than 50% of the cells. No nuclear staining was observed in any of the tissues. | | | |

TABLE 4
HD Tumor TMA for VB6-170

| | Membrane | Score | |
|---|---|---|---|
| Tissue | Staining | Range* | Comments |
| Breast | 8/9 | 1-2+ (4) | 1+ cytoplasmic staining |
| Lung | 7/9 | 1-2+ (2) | 1+ cytoplasmic staining, nuclear staining (rare) |
| Prostate | 7/9 | 1-2+ (2) | 1+ cytoplasmic staining, nuclear staining (very rare) |
| Colon | 6/9 | 1+ | 1+ cytoplasmic staining |
| Ovary | 6/9 | 1+ | 1+ cytoplasmic staining, nuclear staining (very rare) |
| Pancreas | 3/9 | 1+ | 1+ cytoplasmic staining, nuclear staining (very rare) |
| Head & Neck | 2/9 | 1+ | 1+ cytoplasmic staining, nuclear staining (rare) |
| Skin | 2/9 | 1+ | 1+ cytoplasmic staining, nuclear staining (rare) |
| Liver | 1/9 | 1+ | 1+ cytoplasmic staining, nuclear staining (rare) |
| Kidney | 0/9 | 0 | 1+ cytoplasmic staining, nuclear staining (rare) |
| *Slides were scored on a 0-3+ scale, with 0 = no staining and trace being less than 1+ but greater than 0. Grades 1+ to 3+ represented increased intensity of staining, with 3+ being strong, dark brown staining. The concentration of VB6-170 used was 5 μg/mL. For cells staining 1+ the staining was, generally, | | | |
| # focal with less than 10% of the cells staining. For cells staining 2+, staining was observed in greater than 50% of the cells. When observed cytoplasmic staining was usually in 100% of the cells. Numbers in parentheses indicate the number of patients showing 2+ scoring. | | | |

TABLE 5
Summary of the Western blot data for seven cell lines tested

| Cell lines | Antigen | Flow data | WB data |
|---|---|---|---|
| SKOV-3 | 70 + 3 kDa | 54.08 | ++++ |
| DU-145 | 70 + 3 kDa | 48 | +++ |
| A-549 | 70 + 3 kDa | 26 | +++ |
| MB231 | 70 + 3 kDa | 19 | + |
| PC-3 | 70 + 3 kDa | 16.75 | + |
| DAUDI | — | 1 | − |
| C-33A | — | 1 | − |

TABLE 6
Sequences of FR regions

| V_{H} | Framework1 | Framework 2 |
|---|---|---|
| EJ212/076-Cl10 | QVQLVESGGGLVKPGGSLRLSCAASGFTFS | WVRQAPGKGLEWVS |
| VB2-169 | QVQLVESGGGLVKPGGSLRLSCAASGFTFS | WIRQAPGKGLEWVS |
| VB2-170 | QVQLVESGGGLVKPGGSLRLSCAASGFTFS | WVRQAPGKGLEWVS |
| VB2-187 | QVQLVQSGGGLVKPGGSLRLSCAASGFTFS | WVRQAPGKGLEWVS |
| V_{H} | Framework 3 | Framework 4 |
| EJ212/076-Cl10 | RFTISRDNAKNSLYLQMNSLRAEDTAVYYCAR | WGQGTLVTVSS |
| VB2-169 | RFTISRDNAKNSLYLQMNSLRAEDTAVYYCAR | WGQGTLVTVSS |
| VB2-170 | RFTISRDNAKNSLYLQMNSLRAEDTAVYYCAR | WGQGTLVTVSS |
| VB2-187 | RFTISRDNAKNSLYLQMNSLRAEDTAVYYCAR | WGQGTLVTVSS |
| V_{L} | Framework1 | Framework 2 |
| EJ212/076-Cl10 | SYVLTQPPSASGTPGQRVTISC | WYQQLPGTAPKLLIY |
| VB2-169 | SYVLTQPPSASGTPGQRVTISC | WYQQLPGTAPKLLIY |
| VB2-170 | SYELTQPPSASGTPGQRVTISC | WYQQLPGAAPKLLIY |
| VB2-187 | QPVLTQPPSASGTPGQRVTISC | WYQQLPGTAPKLLIY |
| V_{L} | Framework 3 | Framework 4 |
| EJ212/076-Cl10 | GVPDRFSGSKSGTSASLAISGLQSEDEADYYC | FGGGTKLTVL |
| VB2-169 | GVPDRFSGSKSGTSASLAISGLQSEDEADYYC | FGGGTKVTVL |
| VB2-170 | GVPDRFSGSKSGTSASLAISGLQSEDEADYYC | FGGGTKLTVL |
| VB2-187 | GVPDRFSGSKSGTSASLAISGLQSEDEADYYC | FGGGTKLTVL |

TABLE 7

| SEQ | | |
|---|---|---|
| ID | | |
| NO | Description | Sequence |
| 1 | EJ212/076- | |
| | Cl10 na | |
| 2 | EJ212/076- | |
| | Cl10 pp | |
| 3 | VB2-169 na | |
| 4 | VB2-169 pp | |
| 5 | VB2-170 na | |
| 6 | VB2-170 pp | |
| 7 | VB2-187 na | |
| 8 | VB2-187 pp | |
| 9 | | |
| 10 | EJ212/076- | QVQLVESGGGLVKPGGSLRLSCAASGFTFSSYSM |
| | Cl10 VH pp | NWVRQAPGKGLEWVSSISSSSSYIYYADSVKGRF |
| | | TISRDNAKNSLYLQMNSLRAEDTAVYYCARSSGW |
| | | YDGEFDPWGQGTLVTVSS |
| 11 | | |
| 12 | VB2-169 VH pp | QVQLVESGGGLVKPGGSLRLSCAASGFTFSDYYM |
| | | SWIRQAPGKGLEWVSYISSSGSTIYYADSVKGRF |
| | | TISRDNAKNSLYLQMNSLRAEDTAVYYCARSSGW |
| | | YDGEFDPWGQGTLVTVSS |
| 13 | | |
| 14 | VB2-170 VH pp | QVQLVESGGGLVKPGGSLRLSCAASGFTFSSYSM |
| | | NWVRQAPGKGLEWVSSISSSSSYIYYADSVKGRF |
| | | TISRDNAKNSLYLQMNSLRAEDTAVYYCARSSGW |
| | | YDGEFDPWGQGTLVTVSS |
| 15 | | |
| 16 | VB2-187 VH pp | QVQLVQSGGGLVKPGGSLRLSCAASGFTFSSYSM |
| | | NWVRQAPGKGLEWVSSISSSSSYIYYADSVKGRF |
| | | TISRDNAKNSLYLQMNSLRAEDTAVYYCARSSGW |
| | | YDGEFDPWGQGTLVTVSS |
| 17 | | |
| 18 | EJ212/076- | SYVLTQPPSASGTPGQRVTISCSGSSSNIGSNTV |
| | Cl10 VL pp | NWYQQLPGTAPKLLIYSNNQRPSGVPDRFSGSKS |
| | | GTSASLAISGLQSEDEADYYCAAWDDSLNGVVFG |
| | | GGTKLTVL |
| 19 | | |
| 20 | VB2-169 VL pp | SYVLTQPPSASGTPGQRVTISCSGSSSNIGSNTV |
| | | NWYQQLPGTAPKLLIYSNNQRPSGVPDRFSGSKS |
| | | GTSASLAISGLQSEDEADYYCAAWDDSLNAWFGG |
| | | GTKVTVL |
| 21 | | |
| 22 | VB2-170 VL pp | SYELTQPPSASGTPGQRVTISCSGSSSNIGSNTV |
| | | NWYQQLPGAAPKLLIYSNDQRPSGVPDRFSGSKS |
| | | GTSASLAISGLQSEDEADYYCAAWDDSLNGVVFG |
| | | GGTKLTVL |
| 23 | | |
| 24 | VB2-187 VL pp | QPVLTQPPSASGTPGQRVTISCSGSSSNIGSNTV |
| | | NWYQQLPGTAPKLLIYSNNQRPSGVPDRFSGSKS |
| | | GTSASLAISGLQSEDEADYYCAAWDDSLNGWFGG |
| | | GTKLTVL |
| 25 | V_{L} CDR 1 | SGSSSNIGSNTVN |
| 26 | V_{L} CDR 2 | SNXQRPS |
| | (cons) | |
| 27 | V_{L} CDR 2 (1) | SNNQRPS |
| 28 | V_{L} CDR 2 (2) | SNDQRPS |
| 29 | V_{L} CDR 3 | AAWDDSLNXVV |
| | (cons) | |
| 30 | V_{L} CDR 3 (1) | AAWDDSLNAVV |
| 31 | V_{L} CDR 3 (2) | AAWDDSLNGVV |
| 32 | V_{H} CDR 1 | XYXMX |
| | (cons1) | |
| 33 | V_{H} CDR 1 | D/S Y Y/S M S/N |
| | (cons2) | |
| 34 | V_{H} CDR 1 (1) | DYYMS |
| 35 | V_{H} CDR 1 (2) | SYSMN |
| 36 | VH CDR 2 | XISSSXSXIYYADSVKG |
| | (cons1) | |
| 37 | VH CDR 2 | Y/S ISSS S/G S T/Y IYYADSVKG |
| | (cons2) | |
| 38 | VH CDR 2 (1) | YISSSGSTIYYADSVKG |
| 39 | VH CDR 2 (2) | SISSSSSYIYYADSVKG |
| 40 | VH CDR 3 | SSGWYDGEFDP |

TABLE 8

| | | | |
|---|---|---|---|
| Observed | Start | End | Peptide |

| | | | |
|---|---|---|---|
| 529.64 | 79 | 83 | K.NGLVK.I |
| 630.74 | 105 | 109 | K.EELLK.V |
| 651.76 | 99 | 104 | K.FTGLSK.E |
| 684.79 | 150 | 155 | R.ELPAQK.W |
| 928.06 | 110 | 118 | K.VAGSPGWVR.T |
| 1189.34 | 156 | 164 | K.WWHTGALYR.I |
| 1245.31 | 86 | 98 | K.VAEDEAEAAAAAK.F |
| 1328.44 | 50 | 60 | K.EVELNELEPEK.Q |
| 1704.93 | 61 | 78 | K.QPMNAASGAAMSLAGAEK.N |
| 1837.16 | 1 | 17 | .MELQPPEASIAVVSIPR.Q |
| 1867.09 | 165 | 183 | R.IGDLQAFQGHGAGNLAGLK.G |
| 2711.44 | 121 | 144 | R.WALLLLFWLGWLGMLAGAVVIIVR.A |
| 2958.404 | 501 | 530 | FLVVLNFGDVGLSAGLQASDLPASASLPAK |
| 2357.686 | 312 | 334 | LLIAGTNSSDLQQILSLLESNK |
| 2292.582 | 473 | 493 | SLLHGDFHAFSAGPGLFSYIR |

List of peptides along with their respective calculated masses obtained after the reconstruction step is as given in the above table. A total of 15 peptides were recovered in the process.

1. An antibody which comprises at least one heavy chain variable region (VH) that comprises three CDRs, wherein said heavy chain variable region comprises:
(i) a heavy chain CDR1 domain that comprises the amino acid sequence SYSMN (SEQ ID NO. 35),
(ii) a heavy chain CDR2 domain that comprises the amino acid sequence SISSSSSYIYYADSVKG (SEQ ID NO. 39), and
(iii) a heavy chain CDR3 domain that comprises the amino acid sequence SSGWYDGEFDP (SEQ ID NO: 40),
wherein said antibody is capable of specifically binding to CD98hc or fragments of CD98hc.

2. The antibody according to claim 1, which additionally comprises at least one light chain variable region (VL) that comprises three light chain CDRs, wherein one or more of said light chain CDRs is selected from the group consisting of:
(a) a light chain CDR3 domain comprising the amino acid sequence AAWDDSLNGVV (SEQ ID NO:31);
(b) a light chain CDR2 domain comprising the amino acid sequence SNNQRPS (SEQ ID NO:27) or SNDQRPS (SEQ ID NO:28); and
(c) a light chain CDR1 domain comprising the amino acid sequence SGSSSNIGSNTVN (SEQ ID NO. 25).

3. The antibody according to claim 1, which additionally comprises at least one light chain variable region (VL) that comprises three light chain CDRs, wherein two or more of said light chain CDRs are selected from the group consisting of:
(a) a light chain CDR3 domain comprising the amino acid sequence AAWDDSLNGVV (SEQ ID NO:31);
(b) a light chain CDR2 domain comprising the amino acid sequence SNNQRPS (SEQ ID NO:27) or SNDQRPS (SEQ ID NO:28); and
(c) a light chain CDR1 domain comprising the amino acid sequence SGSSSNIGSNTVN (SEQ ID NO. 25).

4. The antibody according to claim 1, which additionally comprises at least one light chain variable region (VL), wherein said light chain variable region comprises one of each of the following light chain CDRs:
(a) a light chain CDR3 domain comprising the amino acid sequence AAWDDSLNGVV (SEQ ID NO:31);
(b) a light chain CDR2 domain comprising the amino acid sequence SNNQRPS (SEQ ID NO:27) or SNDQRPS (SEQ ID NO:28); and
(c) a light chain CDR1 domain comprising the amino acid sequence SGSSSNIGSNTVN (SEQ ID NO. 25).

5. The antibody according to claim 1, which additionally comprises a light chain variable region (VL), wherein said light chain variable region comprises the amino acid sequence given in SEQ ID NO: 18, 22 or 24.

6. The antibody according to claim 1, wherein said heavy chain variable region (VH) comprises the amino acid sequence of SEQ ID NO: 10, 14 or 16.

7. The antibody according to claim 1, wherein said heavy chain variable region (VH) comprises the amino acid sequence of SEQ ID NO: 10, and wherein said antibody additionally comprises at least one light chain variable region (VL) that comprises the amino acid sequence of SEQ ID NO: 18.

8. The antibody according to claim 1, wherein said heavy chain variable region (VH) comprises the amino acid sequence of SEQ ID NO: 14, and wherein said antibody additionally comprises at least one light chain variable region (VL) that comprises the amino acid sequence of SEQ ID NO: 22.

9. The antibody according to claim 1, wherein said heavy chain variable region (VH) comprises the amino acid sequence of SEQ ID NO: 16, and wherein said antibody additionally comprises at least one light chain variable region (VL) that comprises the amino acid sequence of SEQ ID NO: 24.

10. The antibody according to claim 1 comprising the amino acid sequence of SEQ ID NO: 2.

11. The antibody according to claim 1 comprising the amino acid sequence of SEQ ID NO: 6.

12. The antibody according to claim 1 comprising the amino acid sequence of SEQ ID NO: 8.

13. The antibody according to claim 1, wherein said antibody is a human protein.

14. The antibody according to claim 1, wherein said antibody is or comprises an antibody fragment that displays antigen binding function.

15. The antibody according to claim 14, wherein said antibody fragment is selected from the group comprising Fab, Fab′, F(ab′)₂, scFv, Fv, dsFv, ds-scFv, Fd, dAbs, TandAbs dimers, minibodies, diabodies and bispecific antibody fragments.

16. The antibody according to claim 1, wherein said antibody is capable of being internalised.

17. An antibody capable of binding to CD98hc or fragments of CD98hc, wherein said antibody can be identified by a method comprising:
(1) incubating a fixed number of tumor cells which express CD98hc or fragments of CD98hc, with a minimal concentration of an antibody as defined in claim 1 (Ab1) that generates maximal binding against the fixed number of tumor cells and measuring median fluorescence of Ab1 (MF_{Ab1});
(2) testing two or more concentrations of a test antibody (Ab2) by adding Ab2 to the Ab1 and tumor cells, and measuring median fluorescence (MF_{(Ab1+Ab2));}
(3) measuring background median fluorescence (MF_{bgd});
(4) calculating PI, wherein
PI=[(MF_{(Ab1+Ab2})−MF_{Bgd})/(MF_{Ab1-}MF_{Bgd})]×100; and
(5) comparing the PI to a control PI value;
wherein, a PI that has a statistically significant difference from the control PI indicates that the test antibody is capable of binding to CD98hc or fragments of CD98hc.

18. A nucleic acid molecule comprising a sequence encoding an antibody according to claim 1.

19. The nucleic acid molecule according to claim 18, wherein said nucleic acid molecule is as defined in any one of SEQ ID NOs: 1, 5 or 7.

20. A recombinant expression vector comprising one or more nucleic acid molecules according to claim 18.

21. A host cell comprising one or more recombinant expression vectors according to claim 20 and/or one or more of the nucleic acid molecules according to claim 18.

22. A method of producing an antibody as defined in claim 1, comprising a step of culturing the host cells as defined in claim 21.

23. A method according to claim 22, comprising the steps of
(i) culturing said host cell under conditions suitable for the expression of the antibody; and optionally
(ii) isolating said antibody from the host cell or from the growth medium/supernatant; and further optionally
(iii) purification of the antibody product and/or formulating the antibody product into a composition including at least one additional component.

24. An antibody conjugate, wherein an antibody according to claim 1 is conjugated to a label or other detectable moiety, or is conjugated to a biologically active molecule or medically relevant agent.

25. The antibody conjugate of claim 24, wherein said biologically active molecule or medically relevant agent is selected from the group comprising toxins, enzymes, drugs, pre drugs, pro drugs, other small molecule compounds, and nucleic acid molecules.

26. A composition comprising the antibody according to claim 1 or the antibody conjugate according to claim 24, optionally together with one or more pharmaceutically acceptable excipients, carriers, diluents, buffers or stabilizers.

27. A method of diagnosing disease in a mammal comprising the steps of
(1) contacting a test sample taken from said mammal with any one or more of the antibodies according to claim 1 and/or one or more of the antibody conjugates according to claim 24;
(2) measuring the presence and/or amount and/or location of antibody-antigen complex or antibody conjugate-antigen complex in the test sample, wherein the antigen is CD98hc; and, optionally
(3) comparing the presence and/or amount and/or location of antibody-antigen complex or antibody conjugate-antigen complex in the test sample to a control.

28. A method of treatment of a subject comprising the administration of an effective amount of an antibody according to claim 1 and/or an antibody conjugate according to claim 24 to the subject, or to a sample removed from the subject and which is subsequently returned to the subject.

29. The method according to claim 28, wherein the treatment is treatment of cancer.

30. The method according to claim 29, wherein the cancer is selected from the group comprising cervical cancer, uterine cancer, ovarian cancer, pancreatic cancer, kidney cancer, gallbladder cancer, liver cancer, head and neck cancer, squamous cell carcinoma, gastrointestinal cancer, breast cancer, prostate cancer, testicular cancer, lung cancer, non-small cell lung cancer, non-Hodgkin's lymphoma, multiple myeloma, leukemia, brain cancer, neuroblastoma, sarcomas, colon cancer, rectum cancer, stomach cancer, bladder cancer, pancreatic cancer, endometrial cancer, plasmacytoma, lymphoma, and melanoma.

31. A method of diagnosis or imaging of a subject comprising the administration of an amount of the antibody according to claim 1 and/or the antibody conjugate according to claim 24 to the subject and detecting the presence and/or amount and/or the location of said antibody or antibody conjugate.

32. The method according to claim 31, wherein the diagnosis or imaging relates to cancer.

33. A kit comprising:
(i) one or more antibodies according to claim 1 or one or more antibody conjugates according to claim 24, and/or
(ii) one or more nucleic acid molecules according to claim 18, and/or
(iii) one or more recombinant expression vectors or host cells according to any one of claims 20 or 21, and/or
(iv) one or more compositions as defined in claim 26.

34. A method of producing an antibody which is capable of specifically binding to CD98hc or fragments of CD98hc, said method comprising the steps of:
a) modifying one or more of the amino acids in the amino acid sequence of an antibody according to claim 1;
b) testing the resulting modified antibody for the ability to specifically bind to CD98hc or fragments of CD98hc; and
c) selecting a modified antibody which is capable of specifically binding to CD98hc or fragments of CD98hc.
